SEQUENCE LISTING (17 sequences)

SEQ ID NO: 1 - protein, 33 aa, Artificial Sequence
  A synthetic peptide
TERRRQQLDKDGDGTIDEREIKIWFQNKRAKIK

SEQ ID NO: 2 - protein, 33 aa, Artificial Sequence
  A synthetic peptide
TERRRQQLDKDGDGTIDEREIKIHFQNKRAKIK

SEQ ID NO: 3 - protein, 34 aa, Artificial Sequence
  A synthetic peptide
TERRRFDKDGNGYISAAELRHVKIWFQNKRAKIK

SEQ ID NO: 4 - protein, 33 aa, Artificial Sequence
  A synthetic peptide
TERRRFRVFDKDGNGYISAAEKIWFQNKRAKIK

SEQ ID NO: 5 - protein, 33 aa, Artificial Sequence
  A synthetic peptide
TRRRRFLSFDKDGDGTITTKEEVWFQNRRMKWK

SEQ ID NO: 6 - protein, 63 aa, Artificial Sequence
  A synthetic peptide
DEKRPRTAFSGEQLARLKREFNENRYLTERRRLRVFDKDGNGFISAAEKIWFQNKRAKIKKST

SEQ ID NO: 7 - protein, 33 aa, Artificial Sequence; identical to SEQ ID NO: 1
  A synthetic peptide

SEQ ID NO: 8 - protein, 27 aa, Artificial Sequence
  A synthetic peptide
TERRRQQLSSEVGMTCSGCSGQIKIWF

SEQ ID NO: 9 - protein, 24 aa, Artificial Sequence
  A synthetic peptide
TERRRHELMHAIGFYHEAQIKIWF

SEQ ID NO: 10 - protein, 26 aa, Artificial Sequence
  A synthetic peptide
TERRRQQLDKDGDGTIDEREQIKIWF

SEQ ID NO: 11 - protein, 66 aa, Artificial Sequence
  A synthetic peptide
GAMANDEKRPRTAFSSEQLARLKREFNENRYLTERRRDIDGDGTITAKEKIWFQNKRAKIKKSTRV

SEQ ID NO: 12 - protein, 74 aa, Artificial Sequence
  A synthetic peptide
GAMANDEKRPRTAFSSEQLARLKREFNENRYLTERRRQQLFDIDGDGTITAKEEAQIKIWFQNKRAKIKKSTRV

SEQ ID NO: 13 - protein, 21 aa, Artificial Sequence
  A synthetic peptide
TERRRDIDGDGTITAKEKIWF

SEQ ID NO: 14 - protein, 29 aa, Artificial Sequence
  A synthetic peptide
TERRRQQLFDIDGDGTITAKEEAQIKIWF

SEQ ID NO: 15 - protein, 28 aa, Artificial Sequence
  A synthetic peptide
TERRRQQLSHGGGWGEAQIKIWFQNKRA

SEQ ID NO: 16 - protein, 33 aa, Artificial Sequence
  A synthetic peptide
ERRRQQLSSEAETIFGDGDKDEIKIWFQNKRAK

SEQ ID NO: 17 - DNA, 14 bp, Artificial Sequence
  A synthetic oligonucleotide
gagctaattagctc

(19) United States
(12) Patent Application Publication
(10) Pub. No.: US 2005/0112064 A1
(43) Pub. Date: May 26, 2005

(54) DNA-dependent MRI contrast agents

(76) Inventor: Sonya Franklin, Iowa City, IA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/916,943

(22) Filed: Aug. 12, 2004

Related U.S. Application Data
(60) Provisional application No. 60/494,657, filed on Aug. 12, 2003.

(51) Int. Cl. A61K 49/00; C12Q 1/68

(52) U.S. Cl. 424/9.32; 435/6; 530/400

(57) ABSTRACT

The present invention provides magnetic resonance imaging contrast agents comprising a synthetic peptide or polypeptide, and methods to use those agents.

### CROSS-REFERENCE TO RELATED APPLICATIONS

This application claims the benefit of the filing date of U.S. application Ser. No. 60/494,657, filed Aug. 12, 2003, the disclosure of which is incorporated herein by reference.

### STATEMENT OF GOVERNMENT RIGHTS

The invention was made at least in part with a grant from the Government of the United States (grant CHE-0093000 from the National Science Foundation). The Government may have certain rights to the invention.

### BACKGROUND OF THE INVENTION

Molecular imaging has been defined as the in vivo characterization and measurement of biologic processes at the cellular and molecular level (Weissleder et al., 2001). Magnetic resonance imaging (MRI) has great potential to play a broader role in this field, if the poor sensitivity of MRI contrast agents can be overcome. A molecular imaging MRI contrast agent must therefore combine a targeting vector (something that recognizes a specific biomarker) with a high relaxivity entity (a paramagnetic group that makes the interaction detectable by MR). Several different approaches to high relaxivity have been reported, including targeted superparamagnetic particles (Wunderbaldinger et al., 2000); targeted gadolinium-containing emulsions (Moats et al., 1997) and complex assemblies (Nivorozhkin et al., 2001); and enzyme amplified contrast strategies (Louie et al., 2000). For example, Sovoboda et al. (1990) and DeJong et al. (1999) report the preparation of a targeted paramagnetic complex which includes a Gd(III) chelate covalently linked to CCK-8, an eight amino acid peptide that is internalized through the colecystolamine receptor, and Aime et al. (2003) disclose a Gd(III)-loaded liposome which is conjugated to avidin and Gd(III)-loaded apoferritin.

Receptor induced magnetization enhancement (RIME) is an effective MRI signal amplification strategy (Lauffer, 1991; Caravan et al., 2002). When a contrast agent binds to a receptor, the reorientation time (τ_{R}) increases, thereby increasing the relaxation enhancement of solvent proton relaxation rates (1/T₁). Targeted MRI contrast agents that utilize RIME are typically classical bifunctional molecules: a Gd(III) chelate complex appended to a targeting moiety through a tether (FIG. 1). However, the linker group in the bifunctional molecules may introduce rotational flexibility and limit the gain in relaxivity (r₁=(Δ1/T₁)/[Gd]) brought about by receptor binding.

Thus, there is a need for an improved targeted MRI contrast agent.

### SUMMARY OF THE INVENTION

The invention provides a magnetic resonance imaging contrast agent including a synthetic peptide or polypeptide having domains that specifically bind nucleic acid and one or more domains that specifically bind a paramagnetic metal, wherein at least one domain that specifically binds the paramagnetic metal is between domains that specifically bind nucleic acid. The magnetic resonance imaging contrast agent of the invention may translocate into cells, i.e., cross cell membranes and preferably localize in the nucleus. The synthetic peptide or polypeptide exploits the specificity achieved by DNA binding proteins to deliver a paramagnetic metal to a cell or tissue. In particular, the present invention employs a chimeric motif comprising domains based on those found in proteins in nature (“biological domains” of parent proteins), including nucleic acid binding domains and a metal binding domain. In one embodiment, the magnetic resonance imaging contrast agent includes a cell- or subcellular molecule-specific targeting moiety, e.g., a protein such as an antibody or a fragment thereof or protein transport domain, which may be linked to the synthetic peptide or polypeptide or which forms part of a delivery vehicle, for instance, a capsule (e.g., a biodegradable microparticle or nanoparticle in which the targeting moiety is embedded or attached) or matrix in which the synthetic peptide or polypeptide and optionally metal are encapsulated or embedded.

In one embodiment of the invention, DNA binding domains of a parent DNA binding polypeptide, the nucleic acid binding domains of the synthetic peptide or polypeptide of the invention, a metal binding domain of a parent metal binding polypeptide, and the metal binding domain of the synthetic peptide or polypeptide of the invention, have similarity (are substantially superimposable) in their helix orientation. Preferably, at least the turn in the DNA binding domain is substantially superimposable with the metal binding domain. For example, crystal structures of loops that bind metals which have about a 90°, e.g., a 85° to 95°, and preferably a 86° to 94°, turn can be compared to crystal structures of DNA binding domains, e.g., those which have an angle between 60° and 120°, e.g., between 70° and 110°. Thus, a synthetic peptide or polypeptide of the invention has the DNA binding and metal binding properties of its parents, and an angle between 60° and 120°, e.g., between 70° and 110°. In one embodiment, the metal binding domain is about 3 to about 20, or any integer between 3 and 20, e.g., about 4 to about 15, amino acid residues in length. In one embodiment, the metal binding domain has at least 2 and up to 8 residues which donate ligands to a metal. The metal binding domain may bind any suitable metal. In one embodiment, the metal binding domain binds metals including but not limited to Ca(II), Eu(III), Zn(II), Cr(IV), Cd(II), Ce(III), Ce(IV), Fe(III), Co(III), or Cu(II). The metal binding domain may comprise a Lewis-acid metal ion binding motif or in one embodiment comprise a lanthanide-binding motif. For instance, the metal binding domain may be the Ca-binding domain of EF-Hand, the Fe-binding domain of rubridoxin, the Zn-binding domain of astacin, or the Cu-binding domain of Atx1, a copper chaperone protein.

The nucleic acid binding domains each contain at least about 4 amino acids, and together include about 8 to about 50, or any integer in between, for instance, about 12 to about 45, amino acid residues. The nucleic acid binding domains may be from a particular parent polypeptide, which domains may be in close proximity in the primary structure, i.e., within about 50 to about 100 residues of each other or within 500 Angstroms of each other, of that polypeptide, or alternatively are distant from one another in the primary structure of the polypeptide but are in close proximity in a properly folded corresponding protein. In another embodiment, the two nucleic acid domains are from different sources, e.g., from two different DNA binding proteins. The nucleic acid binding domains may be any amino acid sequence which specifically binds a nucleic acid sequence, e.g., a sequence present in dsDNA, dsRNA, ssDNA, ssRNA, A-DNA, B-DNA, Z-DNA and the like. In one embodiment, the nucleic acid binding domains are from a transcription factor, e.g., a homeodomain. Exemplary sources of nucleic acid binding domains include, but are not limited to a domain in genes encoding TTF1, MATα2, TGIF, Antennapedia, Engrailed, Oct-1, Cut, Dlx, e.g., DLX 1-6, Emx, e.g., Emx 1-2, En, e.g., En 1-5, Hox, e.g., Hox 1-11, Hoxa-1-4 and Hoxb-1, Lim, e.g., Lhx1-5, Msx, e.g., Msx1-3, Otx/Otp, e.g., Otx1-2, Pax, e.g., Pax 1-9, Pou, Six/sine oculis, TALE, e.g., Meis2 and TGIF, zinc finger proteins, Barx1-2, Evx1-2, Gtx, Gsh, Lbx, Proxi, Tlx-1, Q50, Eve, Ftz, Ubx, Prd, as well as genes disclosed in Gehring et al. (1994) and Patikoglou et al. (1997), the disclosures of which are specifically incorporated by reference herein (see also FIG. 2). An exemplary winged-helix is that of hepatocyte nuclear factor-38. Preferably, the synthetic peptide or polypeptide of the invention comprises a helix-turn-helix, a winged helix-turn-helix, a relaxed helix-turn-helix or a helix-loop-strand, wherein the turn or loop specifically binds a metal, e.g., binds Ca, any lanthanide, any transition metal including Zn, Mo, Tc, Au, Rh, Ru or W.

As described herein, a high relaxivity Gd(III) chelate was prepared by introducing a metal binding domain within transcription factor DNA binding domains comprising the helical regions of a helix-turn-helix (HTH) domain. Thus, the metal binding domain of the chelate included the topologically (i.e., geometrically) equivalent Ca-binding EF-Hand loop motif at the turn. Moreover, the relaxivity of the chelate was further amplified by binding to DNA, e.g., with a 100% increase in relaxivity.

Exemplary synthetic peptides or polypeptides include SEQ ID NO:3 (P4a, which includes α2 and α3 of engrailed, minus the last turn of α2 and the β-turn, and calmodulin loop I, and incorporates a greater fraction of the EF-Hand turn than P4 and P5; TERRRFDKDGNGYISAAELRHVKIWFQNKRAKIK), SEQ ID NO:4 (P4; TERRRFRVFDKDGNGYISAAEKIWFQNKRAKIK), SEQ ID NO:5 (P5, which includes α2 and α3 of the Antennapedia homeodomain and calmodulin loop III; TRRRRFLSFDKDGDGTITTKEEVWFQNRRMKWK), SEQ ID NO:6 (CMI), SEQ ID NO:7 (PW3, which has a single amino acid substitution relative to P3), SEQ ID NO:8 (P6; TERRRQQLSSEVGMTCSGCSGQIKIWF), SEQ ID NO:9 (P7, which includes a Cu-binding loop from Atx1; TERRRHELMHAIGFYHEAQIKIWF), SEQ ID NO:10 (P3a, which has two amino acid substitutions relative to P3; TERRRQQLDKDGDGTIDEREQIKIWF), SEQ ID NO:11 (P8; GAMANDEKRPRTAFSSEQLARLKREFNENRYLTERRRDIDGDGTITAKE KIWFQNKRAKIKKSTRV), SEQ ID NO:12 (P9; GAMANDEKRPRTAFSSEQLARLKREFNENRYLTERRRQQLFDIDGDGTIT AKEEAQIKIWFQNKRAKIKKSTRV), SEQ ID NO:13 (TERRRDIDGDGTITAKEKIWF), SEQ ID NO:14 (TERRRQQLFDIDGDGTITAKEEAQIKIWF), SEQ ID NO:15 (P7, TERRRQQLSHGGGWGEAQIKIWFQNKRA, which has a Cu binding loop from prion protein), a derivative thereof or an analog thereof. In one embodiment of the invention, the synthetic peptide is not P3, P3W, P3a, P4, P4a, P5, P6, P7, SEQ ID NO:13 or SEQ ID NO:14.

In one embodiment, the synthetic peptide or polypeptide includes a parental helix-turn-helix motif where the turn and optionally a portion of the first helix in the synthetic peptide or polypeptide is a domain which specifically binds a metal, or a portion (fragment) thereof which specifically binds nucleic acid and a metal. In another embodiment, the synthetic peptide or polypeptide includes a parental helix-turn-helix motif where the turn in the synthetic peptide or polypeptide is a loop from a helix-loop-strand domain which specifically binds a metal, e.g., the DNA binding domain of a transcription factor and the metal binding domain of a polypeptide such as Atx1, which binds Cu²⁺, or astacin, which binds Zn²⁺. A peptide or polypeptide of the invention includes at least 20, preferably at least 30, up to 50 or more, e.g., 250, 500 or 1000 or more, residues in length. A peptide is generally less than 50 residues in length.

The invention also provides a method for obtaining a magnetic resonance image (MRI) of a mammal. The method includes administering to the mammal an effective amount of a magnetic resonance imaging contrast agent in a physiologically acceptable medium. The magnetic resonance imaging contrast agent includes a synthetic peptide or polypeptide having domains that specifically bind nucleic acid and one or more domains that specifically bind a paramagnetic metal, wherein one domain that specifically binds the paramagnetic metal is between the domains that specifically bind nucleic acid. The magnetic resonance image of the mammal is then recorded.

The present invention also provides a method for making magnetic resonance measurements of a sample of mammalian tissue by modifying the characteristic relaxation times of water protons in the sample. The method includes introducing to a sample of mammalian tissue a magnetic resonance imaging contrast agent comprising a synthetic peptide or polypeptide having domains that specifically bind nucleic acid and one or more domains that specifically bind a paramagnetic metal, wherein one domain that specifically binds the metal is between the domains that specifically bind nucleic acid. The sample is then placed in a magnetic field and subjected to a radio frequency pulse. The relaxation times of the sample are then measured.

Another embodiment of the invention provides a method for enhancing contrast in magnetic resonance images of a sample of mammalian tissue. The method includes introducing to a sample of mammalian tissue a magnetic resonance imaging contrast agent comprising a synthetic peptide or polypeptide having domains that specifically bind a nucleic acid and one or more domains specifically bind a paramagnetic metal, wherein one domain that specifically binds the metal is between the domain that specifically binds nucleic acid. Magnetic resonance imaging of the sample is then performed.

### BRIEF DESCRIPTION OF THE FIGURES

FIG. 1. A bifunctional contrast agent having a Gd-chelate and targeting moiety tethered by a linker.

FIG. 2A. Stereo view of the overlay of engrailed HTH region (α2-α3; 1ENH) and one EF-Hand of parvalbumin (5PAL), to illustrate that the helical axes are colinear. The C-terminal α3 is the homeodomain DNA-recognition helix. Engrailed is shown in green, parvalbumin in blue, and the Ca(II) ion with its ligands in magenta.

FIG. 2B. A side by side stereo view of DNA and a DNA (green) and metal binding (magenta) peptide (metal is shown in blue).

FIG. 2C. Two views of the overlay of engrailed (1ENH) helix-turn-helix (HTH) region (α2-α2) and one EF-hand of calmodulin (1OSA; Ca-site) thus illustrating that the helices occupy the same space. The C-terminal α3 is the homeodomain recognition helix which binds in the DNA major grove. The Ca(II) ion is shown as a sphere.

FIG. 3. Sequences of peptide P2 (control; SEQ ID NO:16), P3, P3W, P3a, P4, P4a, P5, P6, P7 (synthetic; SEQ ID NO:2, SEQ ID NO:7, SEQ ID NO:10, SEQ ID NO:4, SEQ ID NO:3, SEQ ID NO:5, SEQ ID NO:8, and SEQ ID NO:9, respectively), P8, P9 and CM1 (synthetic loop modified Engrailed, SEQ ID NO:11, SEQ ID NO:12, and SEQ ID NO:6, respectively; loop in P8 and P9 is bolded). P8 and P9 show the entire Engrailed homeodomain with a calmodulin-based loop. Parent protein sequence is indicated by double or single underlining and the 12 residues of the Ca-binding loop are shaded (homeodomain and EF-hand for P2, P3, P3W, P4a, P5 and CM1). Expected sites of Ca(II) binding are indicated by an x.

FIG. 4. A chimeric contrast agent having a Gd-chelate embedded within a targeting moiety.

FIG. 5. ¹H NMRD relaxivities (35° C.) of GdP3W (33 μM) in the presence (filled circles) and absence (open circles) of 1 equivalent of DNA.

FIG. 6. T1-weighted MR images at 1.5 tesla of phantoms in HEPES buffer, pH 7.4, ambient temperature. (A) 3D SPGR TR=40, TE=3.1, α=75°. Images: a) HEPES; b) 80 μM P3W; c) 80 μM DNA (14-mer duplex); d) 115 μM GdDTPA; e) 115 μM GdDTPA+DNA (1:1); f) 8.6 μM GdP3W+DNA (1:1); g) 86 μM GdP3W; h) 86 μM GdP3W+DNA (1:1); i) 86 μM GdP3W+DNA (1:1)+NaCl (150 μM). (B) 3D SPGR TR=40, TE=4.0, α=40°. Images: a) HEPES, b) 48 μM GdDTPA, c) 48 μM GdP3W; d) 48 μM GdP3W+DNA.

FIG. 7. Molar ellipticity data.

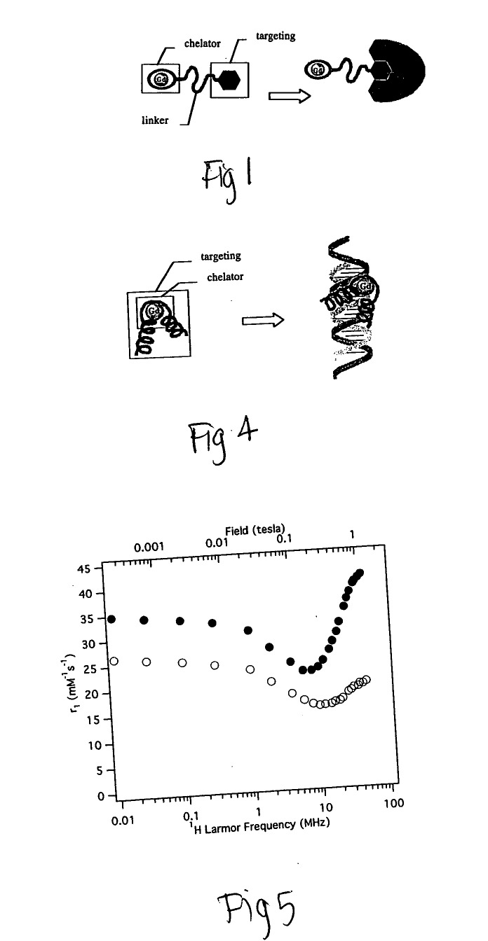

### DETAILED DESCRIPTION OF THE INVENTION

Definitions

A synthetic peptide or polypeptide of the invention, or an analog or a derivative thereof, which specifically binds to nucleic acid and specifically binds a paramagnetic metal, comprises an amino acid sequence which is not found in nature.

A “peptide or polypeptide” includes any molecule having two or more natural or unnatural amino acids linked together, either D or L amino acids, including a peptide or polypeptide which is subjected to chemical modifications, such as esterification, amidation, reduction, protection and the like (“derivatives”). Other “derivatives” of the invention include branched peptides, circular branched and branched circular peptides.

An “analog” of a peptide or polypeptide of the invention is a molecule that mimics the activity of that peptide or polypeptide but which is not a peptide or polypeptide or a derivative thereof. As used herein, the term “mimics” means that the molecule has a similar activity to that of a peptide or polypeptide of the invention, but that the activity of the analog is not necessarily of the same magnitude as the activity of the peptide or polypeptide.

It is also envisioned that the peptides, polypeptides, derivatives and analogs thereof of the invention may comprise moieties other than the portions which bind a nucleic acid sequence or bind a paramagnetic metal, such as an antibody or a fragment thereof, a fusion protein, nucleic acid molecules, sugars, lipids, fats, a detectable signal molecule such as a radioisotope, e.g., gamma emitters, sound wave emitters, small chemicals, metals other than those that bind to the peptide, polypeptide, derivative or analog thereof of the invention, salts, synthetic polymers, e.g., polylactide and polyglycolide, surfactants and glycosaminoglycans, which preferably are covalently attached or linked to the peptide, polypeptide, derivative, or analog thereof, of the invention so that the other moiety does not alter the activity of the peptide, polypeptide, derivative or analog thereof. Also envisioned is a peptide, polypeptide, derivative or analog thereof that is non-covalently associated with the moieties described above.

A “paramagnetic metal” refers to a suitable metal or metal ion useful for diagnostic purposes in the present invention, e.g., a metal with unpaired electrons. Suitable paramagnetic metals include transition elements and lanthanide series inner transition elements. Additional suitable paramagnetic metals include, e.g., Yttrium (Y), Molybdenum (Mo), Technetium (Tc), Ruthenium (Ru), Rhodium (Rh), Tungsten (W), and Gold (Au). Additional specific suitable specific paramagnetic metals include, e.g., Y(III), Mo(VI), Tc(IV), Tc(VI), Tc(VII), Ru(III), Rh(III), W(VI), Au(I), and Au(III).

A “lanthanide,” “lanthanide series element” or “lanthanide series inner transition element” refers to Cerium (Ce), Praseodymium (Pr), Neodymium (Nd), Promethium (Pm), Samarium (Sm), Europium (Eu), Gadolinium (Gd), Terbium (Th), Dysprosium (Dy), Holmium (Ho), Erbium (Er), Thulium (Tm), Ytterbium(Yb), or Lutetium (Lu). Specific suitable lanthanides include, e.g., Ce(III), Ce(IV), Pr(III), Nd(III), Pm(III), Sm(II), Sm(III), Eu(II), Eu(II), Gd(III), Tb(III), Dy(III), Ho(III), Er(III), Tm(III), Yb(II), Yb(III), and Lu(III).

A “first row transition metal” refers to Calcium (Ca), Scandium (Sc), Titanium (Ti), Vanadium (V), Chromium (Cr), Manganese (Mn), Iron (Fe), Cobalt (Co), Nickel (Ni), Copper (Cu), or Zinc (Zn). Specific first row transition metals include, e.g., Sc(II), Ti(II), Ti(III), Ti(IV), V(II), V(III), V(IV), V(V), Cr(II), Cr(III), Cr(VI), Mn(III), Mn(III), Mn(IV), Mn(VI), Fe(II), Fe(III), Co(II), Co(III), Ni(II), Ni(III), Cu(I), Cu(II), and Zn(II).

The term “at least two” refers to two or more (e.g., 2, 3, 4, 5, etc.). Specifically, the range can include between 2 and 10, inclusive; can include between 2 and 8, inclusive; can include between 2 and 6, inclusive; and can include between 2 and 4, inclusive.

An “isolated” nucleic acid molecule, peptide or polypeptide refers to in vitro preparation, isolation and/or purification of a nucleic acid molecule, peptide, or polypeptide of the invention, so that it is not associated with in vivo substances or other molecules present in an in vitro synthesis. Thus, with respect to an “isolated nucleic acid molecule”, which includes a polynucleotide of genomic, cDNA, or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. An isolated nucleic acid molecule means a polymeric form of nucleotides of at least 10 bases in length, either ribonucleotides or deoxynucleotides or a modified form of either type of nucleotide. The term includes single and double stranded forms of DNA. The term “oligonucleotide” referred to herein includes naturally occurring, and modified nucleotides linked together by naturally occurring, and non-naturally occurring oligonucleotide linkages. Oligonucleotides are a polynucleotide subset with 200 bases or fewer in length. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g., for use in the construction of an expression cassette. Oligonucleotides of the invention can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoroaniladate, phosphoroamidate, and the like. An oligonucleotide can include a label for detection, if desired.

The phrase “isolated peptide” or “isolated polypeptide” means a peptide or polypeptide encoded by cDNA or recombinant RNA, or is synthetic in origin, or some combination thereof, which isolated polypeptide or peptide (1) is not associated with proteins found in nature, (2) is free of other proteins from the same source, e.g., free of human proteins, (3) is expressed by a cell from a different species, or (4) does not occur in nature.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of one sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

The term “selectively hybridize” means to detectably and specifically bind. Polynucleotides, oligonucleotides and fragments of the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest is at least 65%, and more typically with preferably increasing homologies of at least about 70%, about 90%, about 95%, about 98%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff (1972). The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide-sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) by the homology alignment algorithm of Needleman and Wunsch (1970) by the search for similarity method of Pearson and Lipman (1988) by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to the sequence of a peptide or a polypeptide, the term “substantial identity” means that two peptide or polypeptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

I. Preparation of Peptides and Polypteptides and Nucleic Acid Molecules of the Invention

In one embodiment of the invention, the three-dimensional structure of a polypeptide that specifically binds nucleic acid and another polypeptide which specifically binds a metal are compared. The comparison may include a visual inspection or computer analysis of the three-dimensional structures including but not limited to crystal structures such as those available in the Swiss Protein Data Bank and NMR structures. The sequences of nucleic acid binding domains and metal binding domains that have geometric similarity (e.g., Efimov, 1994; Efimov, 1996; Efimov, 1997; Richardson et al., 1992) are selected to prepare synthetic peptides or polypeptides. Preferably, the metal binding domain is introduced into, or in place of, a region within a sequence having nucleic acid binding domains which region is not required for binding to nucleic acid. Additional residues may be deleted or inserted in one or more of the domains, or certain residues may be substituted to enhance the properties of the synthetic peptide or polypeptide, properties including but not limited to nucleic acid binding, metal binding, stability, affinity for proteins, membrane translocation, or biodistribution properties.

A. Nucleic Acid Molecules

1. Expression Cassettes

To prepare expression cassettes for transformation herein, the recombinant DNA sequence or segment encoding the peptide or polypeptide of the invention may be circular or linear, double-stranded or single-stranded. A DNA sequence which encodes an RNA sequence that is substantially complementary to a mRNA sequence is typically a “sense” DNA sequence cloned into a cassette in the opposite orientation (i.e., 3′ to 5′ rather than 5′ to 3′). Generally, the recombinant DNA sequence or segment is in the form of chimeric DNA, such as plasmid DNA, that can also contain coding regions flanked by control sequences which promote the expression of the DNA present in the resultant host cell.

As used herein, “chimeric” nucleic acid means that a vector comprises DNA from at least two different species, is synthetic or comprises DNA from the same species which is linked or associated in a manner which does not occur in the “native” or wild type of the species.

Aside from DNA sequences that serve as transcription units, a portion of the recombinant DNA may be untranscribed, serving a regulatory or a structural function. For example, the DNA may itself comprise a promoter that is active in prokaryotic or eukaryotic cells such as mammalian cells, or may utilize a promoter already present in the genome that is the transformation target.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a peptide or polypeptide if it is expressed as a preprotein that participates in the secretion of the peptide or polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may further contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroa, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis. Expression of the reporter gene is assayed at a suitable time after the recombinant DNA has been introduced into the recipient cells.

The general methods for constructing recombinant DNA which can transform target cells are well known to those skilled in the art, and the same compositions and methods of construction may be utilized to produce the DNA useful herein. For example, Sambrook et al. (1989), provides suitable methods of construction.

2. Transformation into Host Cells

The recombinant DNA can be readily introduced into the host cells, e.g., mammalian, bacterial, yeast or insect cells by transfection with an expression vector comprising DNA or its complement, by any procedure useful for the introduction into a particular cell, e.g., physical or biological methods, to yield a transformed cell having the recombinant DNA stably integrated into its genome, so that the DNA molecules, sequences, or segments, of the present invention are expressed by the host cell.

Physical methods to introduce a recombinant DNA into a host cell include calcium phosphate precipitation, lipofection, particle bombardment, microinjection, electroporation, and the like. Biological methods to introduce the DNA of interest into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For mammalian gene therapy, it is desirable to use an efficient means of precisely inserting a single copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As used herein, the term “cell line” or “host cell” is intended to refer to well-characterized homogenous, biologically pure populations of cells. These cells may be eukaryotic cells that are neoplastic or which have been “immortalized” in vitro by methods known in the art, as well as primary cells, or prokaryotic cells. The cell line or host cell may be of mammalian origin or of non-mammalian origin, including plant, insect, yeast, fungal or bacterial sources.

“Transfected” or “transformed” is used herein to include any host cell or cell line, the genome of which has been altered or augmented by the presence of at least one recombinant DNA sequence, which DNA is also referred to in the art of genetic engineering as “heterologous DNA,” “exogenous DNA,” “genetically engineered,” “non-native,” or “foreign DNA,” wherein said DNA was isolated and introduced into the genome of the host cell or cell line by the process of genetic engineering. The host cells of the present invention are typically produced by transfection with a DNA sequence in a plasmid expression vector, a viral expression vector, or as an isolated linear DNA sequence.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular endonuclease, e.g., by immunological means (ELISAs and Western blots) or by assays described herein to identify molecules falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced preselected DNA sequences or evaluating the phenotypic changes brought about by the expression of the introduced recombinant DNA segment in the host cell.

B. Peptides and Polypeptides

The present isolated peptides or polypeptides can be synthesized in vitro, e.g., by the solid phase peptide synthetic method or by recombinant DNA approaches (see above). The solid phase peptide synthetic method is an established and widely used method, which is described in the following references: Stewart et al. (1969); Merrifield (1963); Meienhofer (1973); Bavaay and Merrifield, (1980); and Clark-Lewis et al. (1997). These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, can be readily prepared. For example, amides of the peptide or polypeptide of the present invention may also be prepared by techniques well known in the art for converting a carboxylic acid group or precursor, to an amide. A preferred method for amide formation at the C-terminal carboxyl group is to cleave the peptide from a solid support with an appropriate amine, or to cleave in the presence of an alcohol, yielding an ester, followed by aminolysis with the desired amine.

Salts of carboxyl groups of a peptide or polypeptide of the invention may be prepared in the usual manner by contacting the peptide or polypeptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide or polypeptide may be prepared by utilizing an N-acyl protected amino acid for the final condensation, or by acylating a protected or unprotected peptide or polypeptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N— and O-acylation may be carried out together, if desired.

Formyl-methionine, pyroglutamine and trimethyl-alanine may be substituted at the N-terminal residue of the peptide or polypeptide. Other amino-terminal modifications include aminooxypentane modifications (see Simmons et al., 1997).

In addition, the amino acid sequence of a particular peptide or polypeptide can be modified so as to result in a peptide or polypeptide variant of that particular peptide or polypeptide, e.g., the amino acid sequence of the variant has substantial identity to the reference peptide or polypeptide. The modification includes the substitution of at least one amino acid residue in the peptide for another amino acid residue, including substitutions which utilize the D rather than L form, as well as other well known amino acid analogs, e.g., unnatural amino acids such as α,α-disubstituted amino acids, N-alkyl amino acids, lactic acid, and the like. Amino acid analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, ω-N-methylarginine, and other similar amino acids and imino acids and tert-butylglycine. Conservative amino acid substitutions are preferred—that is, for example, aspartic-glutamic as acidic amino acids; lysine/arginine/histidine as basic amino acids; leucine/isoleucine/methionine/alanine/valine/glycine as hydrophobic amino acids; serine/threonine as hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional peptide or polypeptide can readily be determined by assaying the activity of the peptide or polypeptide variant.

Amino acid substitutions falling within the scope of the invention, are, in general, accomplished by selecting substitutions that do not differ significantly in their effect on maintaining (a) the structure of the peptide or polypeptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

The invention also envisions peptide or polypeptide variants with non-conservative substitutions. Non-conservative substitutions entail exchanging a member of one of the classes described above for another.

Acid addition salts of amino residues of the peptide or polypeptide may be prepared by contacting the peptide or amine with one or more equivalents of the desired inorganic or organic acid, such as, for example, hydrochloric acid. Esters of carboxyl groups of the peptides or polypeptides may also be prepared by any of the usual methods known in the art.

Peptide or polypeptide analogs have properties analogous to those of the corresponding peptide. These analogs can be referred to as “peptide mimetics” or “peptidomimetics” (Fauchere (1986); Veber and Freidinger (1985); and Evans et al. (1987)) and can be developed with the aid of computerized molecular modeling. These analogs include structures having one or more peptide linkages optionally replaced by a linkage selected from the group consisting of: —CH₂NH—, —CH₂S—, —CH₂—CH₂—, —CH═CH-(cis and trans), —CH═CF-(trans), —CoCH₂—, —CH(OH)CH₂, and —CH₂SO—, by methods known in the art and further described in the following references: Spatola (1983); Spatola (1983); Morley (1980); Hudson (1979) (—CH₂NH—, CH₂CH₂—); Spatola (1986) (—CCH₂—S); Hann (1982) (—CH—CH—, cis and trans); Almquist (1980) (—COCH₂—); Jennings-White et al. (1982) (—COCH₂—); EP 45665 (—CH(OH)CH₂—); Holladay et al. (1983) (—C(OH)CH₂—); and Hruby (1982) (—CH₂S—). A particularly preferred non-peptide linkage is —CH₂NH—. Such analogs may have greater chemical stability, enhanced pharmacological properties (half-life, absorption, potency, efficacy, etc.), altered specificity (e.g., a broad-spectrum of biological activities), reduced antigenicity, and be economically prepared. Labeling of analogs usually involves covalent attachment of one or more labels, directly or through a spacer (e.g., an amide group), to non-interfering positions(s) on the analog that are predicted by quantitative structure-activity data and/or molecular modeling. Such non-interfering positions generally are positions that do not form direct contacts with the macromolecule(s) to which the analog specifically binds to produce the desired effect. Systematic substitution of one or more amino acids of a consensus sequence with a D-amino acid of the same type (e.g., D-lysine in place of L-lysine) may also be used to generate more stable peptides.

D. Identification of Synthetic Molecules Falling Within the Scope of the Invention

Once a peptide or polypeptide of the invention is prepared, it may be characterized by methods well known to the art including: 1) comparing the structural similarity of the synthetic molecule to the parent motifs, e.g., comparing the α-helicity of the molecule as a function of metal using CD, the solution structure and folding dynamics in the presence of the metal via 1D- and 2D-NMR and X-ray crystallography, and peptide- or polypeptide-DNA interactions by NMR and X-ray crystallography; 2) determining the affinity of the synthetic peptide or polypeptide for various metals, dimerization, and DNA binding, for example, determining the binding affinity of the metal, equilibrium dialysis, CD thermal melt and calorimetric titrations as a function of metal, and determining the kinetics and thermodynamics of dimerization by 1D-NMR titrations, centrifugal sedimentation, and CD denaturation studies, while PAGE gel-shift and footprinting assays are employed with ³²P-labeled DNA for DNA binding and binding constant determinations; 3) establishing DNA binding, for example, gel-shift assays of ³²P-radiolabeled DNA as a function of metallopeptide or metallopolypeptide to quantify DNA binding; and/or 4) determining the relaxivity of the metallopeptide in the presence and absence of nucleic acid.

For peptides or polypeptides, in particular, for 1), the α-helicity, β-sheet, and random coil content can be estimated from the molar ellipticity between 200 and 230 nm. The secondary fold is studied by NMR to determine the solution structure and dynamics in the presence of metals. 2D-NOESY-COSY, and TOCSY experiments in D₂O and 90:10 H₂O:D₂O are employed to assign peaks, from which both local 2° structure and full 3D-solution structures can be calculated. Further, the effect of the Ca-binding loop structure and sequence on the overall peptide or polypeptide fold is assessed by selected residue modifications coupled to these structural studies.

For 2), metal-binding and dimerization constants (K_{d} and K_{dim}) may be determined in several ways. PAGE gel-shift assays and centrifugal sedimentation allow the K_{dim} to be determined. Isothermal titration microcalorimetry may be used to determine binding affinities and the thermodynamics of peptide or polypeptide dimerization and self-assembly by quantifying heat releases with the addition of aliquots of metal. Alternately, equilibrium dialysis of solutions of varying metal/peptide or polypeptide ratios and K_{dim} can be determined. Substitutions in the loop metal-binding residues may also be made to further determine metal affinities. The amount of stabilization afforded the peptide or polypeptide by metal binding is also investigated by CD thermal denaturation studies. In the presence and absence of metal, changes in the CD as a function of temperature can be correlated to thermodynamic parameters. In addition to these studies, metal binding constants and the number of inner sphere water molecules can be determined for the peptide or polypeptide bound to metal by luminescence titrations.

For 3), the thermodynamics of DNA binding is investigated by gel-shift and footprinting assays of ³²P-radiolabeled DNA as a function of metalated peptide or polypeptide. Oligonucleotides may be ³²P-labeled for acrylamide gel electrophoresis, and binding gels are quantified using Molecular Dynamics PhosphorImager technology.

II. Dosages, Formulations and Routes of Administration of the MRI Contrast Agents of the Invention

The MRI contrast agents of the invention are preferably administered, at dosages of at least about 0.01 to about 0.1, more preferably about 0.02 to about 0.075, and even more preferably about 0.02 to about 0.03 mmol/kg, although other dosages may provide beneficial results. The amount administered will vary depending on various factors including, but not limited to, the agent chosen, the target organ or tissue and if the agent is modified for cell, organ or tissue targeting, bioavailability and/or in vivo stability.

Administration of the agents in accordance with the present invention may be continuous or intermittent, depending, for example, upon the recipient's physiological condition, and other factors known to skilled practitioners. The administration of the agents of the invention may be essentially continuous over a preselected period of time or may be in a series of spaced doses. Both local and systemic administration is contemplated.

One or more suitable unit dosage forms comprising the agents of the invention, which, as discussed below, may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the agent with liquid carriers, solid matrices, semi-solid carriers, finely divided solid carriers or combinations thereof, and then, if necessary, introducing or shaping the product into the desired delivery system.

When the agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Formulations suitable for vaginal administration may be presented as pessaries, tampons, creams, gels, pastes, douches, lubricants, foams or sprays containing, in addition to the active ingredient, such carriers as are known in the art to be appropriate. Formulations suitable for rectal administration may be presented as suppositories.

Pharmaceutical formulations containing the agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The agents of the invention can also be formulated as elixirs or solutions for convenient oral administration or as solutions appropriate for parenteral administration, for instance by intramuscular, subcutaneous or intravenous routes.

The pharmaceutical formulations of the agents of the invention can also take the form of an aqueous or anhydrous solution or dispersion, or alternatively the form of an emulsion or suspension.

Thus, the agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and α-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The agents of the invention can be delivered via patches for transdermal administration. See U.S. Pat. No. 5,560,922 for examples of patches suitable for transdermal delivery of an agent. Patches for transdermal delivery can comprise a backing layer and a polymer matrix which has dispersed or dissolved therein an agent, along with one or more skin permeation enhancers. The backing layer can be made of any suitable material which is impermeable to the agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogen), polyvinylidene chloride, poly(ethylene terephthalate), butyl rubber, epichlorohydrin rubbers, ethylene-vinyl alcohol copolymers, ethylene-vinyloxyethanol copolymers; silicone copolymers, for example, polysiloxane-polycarbonate copolymers, polysiloxane-polyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Agents released from a transdermal delivery system must be capable of penetrating each layer of skin. In order to increase the rate of permeation of an agent, a transdermal drug delivery system must be able in particular to increase the permeability of the outermost layer of skin, the stratum corneum, which provides the most resistance to the penetration of molecules. The fabrication of patches for transdermal delivery of agents is well known to the art.

For administration to the upper (nasal) or lower respiratory tract by inhalation, the agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the agent may be administered via nose drops, a liquid spray, such as via a plastic bottle atomizer or metered-dose inhaler. Typical of atomizers are the Mistometer (Wintrop) and the Medihaler (Riker).

The local delivery of the agents of the invention can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the agents may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms, e.g., via a coated condom. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of an agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The invention will be further described by the following non-limiting examples.

### EXAMPLE 1

The remarkable similarity of the intermolecular interactions of many DNA binding proteins, despite little homology in their primary sequence, suggests that certain motifs are particularly well suited to complement the structure of B-form DNA (Brennan et al., 1989; Burley, 1994; Pabo et al., 1984; Patikoglou et al, 1997). The helix-turn-helix (HTH) motif is a well-known DNA binding motif which complements the shape of the DNA major groove. Homeodomains were first discovered in Drosophila, these small proteins bind DNA and regulate growth and development (Gehring et al., 1994; Kornberg, 1993; Treisman et al., 1992). Homeodomains are transcription factors, either activators or repressors (e.g., Cro and λ repressors) which typically have about 60 residues in the homeodomain arranged as three helices, with the amino terminal arm contacting the minor groove (Burley, 1994; Patikoglou et al., 1997; Laughan, 1991). Homeodomains which comprise the HTH motif can specifically bind to DNA as a monomer or as a dimer which has enhanced affinity relative to a monomer (Freemont et al., 1991). The canonical HTH motif contains two orthogonal helices, spanned by a β-turn four residues in length (Patikoglou et al., 1997), although longer loops rather than β-turns may separate the two helices. The latter of the two HTH helices, the recognition helix, lies in the major groove of DNA allowing side chain contacts to be made to specific base sequences. However, the HTH geometry is not unique to transcription factors, but is in fact a common structural unit in proteins as diverse as Taq polymerase and Cyt c peroxidase.

In homeodomains, the HTH domain both interacts selectively with DNA and promotes translocation through cellular and nuclear membranes. Translocation is apparently an inherent property of these proteins, presumably dependent on the third helix of the HTH motif. This helix (α3) has in fact been shown to be a general membrane translocation vector for an array of hydrophilic cargoes, including non-native peptide sequences and DNA oligonucleotide fragments. The recognition helix of the homeodomain Antennapedia has even been dubbed “penetratin” for its remarkable ability to translocate to the nucleus via a receptor-independent internalization mechanism.

The similarity of the HTH and the Ca-binding EF-Hand structures was striking: these two motifs are variants of the α-α corner described by Efimov as part of his structural tree approach to protein classification (1984; 1986). Efimov concludes that this turn represents an inherently stable fold across a wide variety of non-homologous proteins. Thus, a design which maintains key a-helical regions, and interhelical hydrophobic and hydrophilic contacts, should result in a chimeric construct with a similar α-α corner structure.

Similar to the HTH in topology, but unrelated in function, is the ubiquitous Ca-binding motif, the EF-Hand. The EF-Hand, named for the orthogonal “thumb and forefinger” orientation between a pair of helices, contains a loop that incorporates the Ca(II) binding pocket (Celio et al., 1996). This Ca-binding loop comprises twelve residues, including six highly conserved, mostly acidic residues making side chain or backbone contacts to the metal. The motif commonly occurs in pairs in Ca(II) signaling and regulatory proteins, and in fact, isolated EF-Hand peptides have been found to form discrete dimers in solution (Ca₂loop₂) (Shaw et al., 1991; Wójcik et al., 1997; Clark et al., 1993). The subtle tuning of metal-specificity and cooperativity mediates the biological role of a given EF-Hand protein in signaling pathways. The dissociation constant for Ca(II) from an EF-Hand site is generally on the order of μM, though this varies with loop structure. EF-Hands sites exhibit a 10⁶ fold range in Ca(II) affinity, and up to a 1000-fold preferential specificity over Mg(II).

Lanthanide(III) ions have been successfully substituted into EF-Hands, with coordination geometries essentially identical to that of the native Ca(II) ion (Bruno et al., 1992; Falke et al., 1991; Coruh et al., 1994). Ln(III) ions are very similar in size to Ca(II) ions, and thus bind with higher affinity than Ca(II) due to their larger charge to size ratio. This greater charge density has been suggested to promote increased rigidity of structure in Ln-EF-Hands (Wójcik et al., 1997). Thus by selecting a Ln(III) ion and binding pocket, a sequence with high metal affinity, slow dissociation kinetics, and constrained geometry can be prepared. Further, lanthanide ions have been shown to enhance the rate of phosphate hydrolysis of enzymes by a factor of 10⁷, and provide excellent NMR, fluorescent, and luminescent spectral markers for studying structure and molecular interactions of biomolecules. Both metal-binding and hydrophobic interactions between helices promote the homeodomain's native tertiary structure, and thus its DNA binding function is also maintained.

Materials and Methods

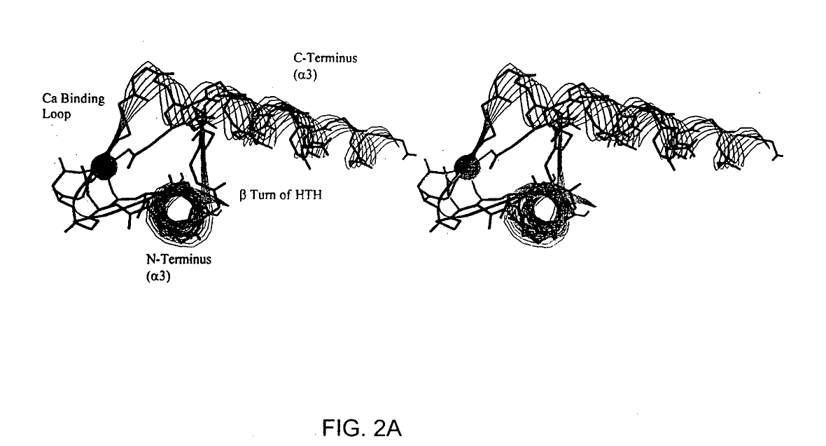

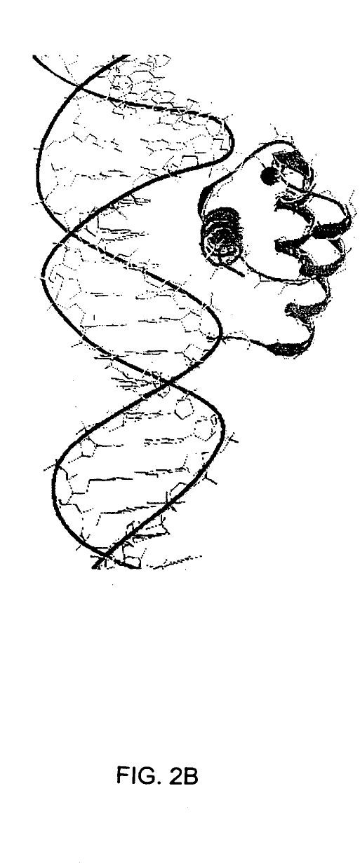

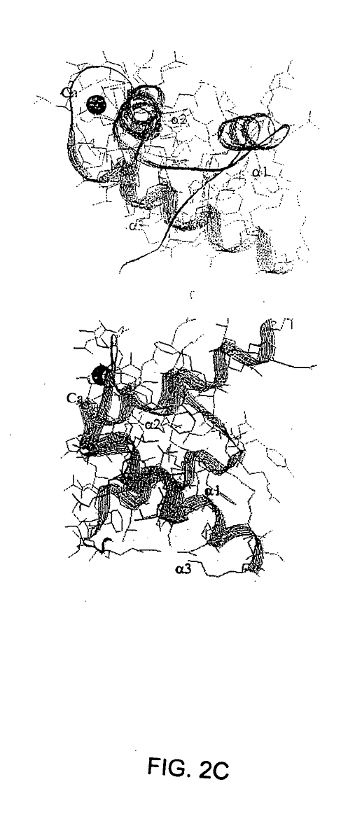

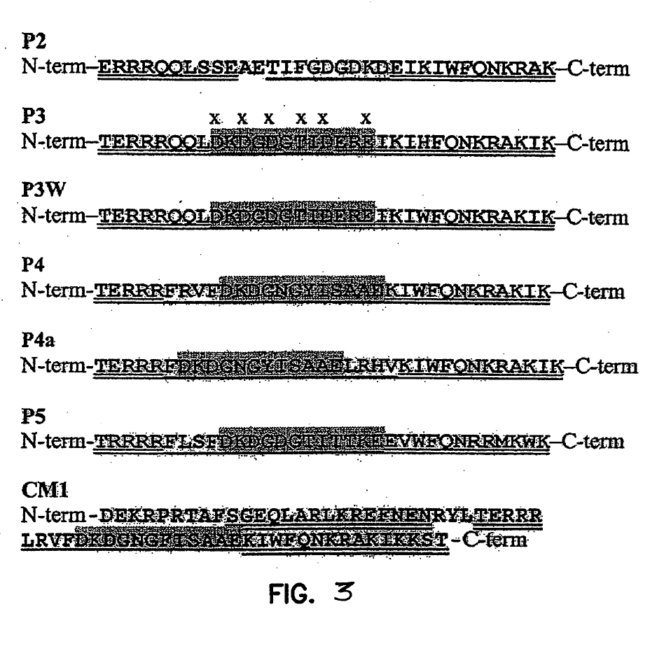

Chimeric Design and Synthesis. The 33-residue peptides shown in FIG. 3B were based on overlays of engrailed and calmodulin crystal structures (FIG. 2B). Known protein crystal structures were oriented manually using the freeware program SwissPDBViewer (Guex et al., 1997) to align the fold of the homeodomain HTH motifs and Ca-binding protein EF-Hand motifs. Crystal coordinates were downloaded from the Protein Data Bank (PDB) for several EF-Hand proteins, such as calmodulin (1OSA) (Chattopadhyaya et al., 1992), parvalbumin (5PAL) (Roquet et al., 1992), and calcineurin (1TCO) (Kissinger et al., 1995). Coordinates for the homeodomain proteins engrailed (with and without co-crystallized DNA, 2HDD and 1ENH, respectively) (Clarke et al., 1994; Kissinger et al., 1990; Tucker-Kellogg et al., 1997) and antennapedia with DNA (9ANT) (Fraenkel et al., 1998) were obtained. The best fits (determined by inspection and RMS deviation of small helical sections) were used.

P3 (SEQ ID NO:2) is a consensus EF-Hand loop, P2 (SEQ ID NO:1) is a reverse EF-Hand loop, and P4a (SEQ ID NO:4) comprises α2 and α3 of engrailed, minus the last turn(s) of α2 and the β-turn, and contains calmodulin loop I (FIG. 3). The EF-Hand and calmodulin loop I motifs have two helices at approximate right angles to one another. P4a incorporates a greater fraction of the EF-Hand turn (single underline in FIG. 2) than does P3, as well as retaining the native salt bridges (Arg-Glu) and hydrophobic contacts (Phe-Phe) between the first turns of helix E and helix F, including an aromatic Tyr group (Y₁₃) in the loop, resulting in a shift in register of the Ca-binding loop to the N-terminal side.

The synthesis of peptides P2 and P3 was done by Dr. Suzanna Horvath of the Caltech Peptide Synthesis Facility, and P4a by Anaspec, Inc. The peptides were synthesized by Fmoc chemistry, cleaved from the resin, and HPLC purified to >95% purity. Concentrations of stock solutions were determined by Bradford assay (Bradford, 1976). EuCl₃ stock solutions were prepared by weight from EuCl₃.(H₂O)₆ (Aldrich, 99.99%), and adjusted to pH=6.0 with dilute NaOH.

Circular Dichroism Titrations. The titration of P2 and P3 peptides with Eu(III) and Ca(II) was followed by circular dichroism spectroscopy on an Aviv 6ODS spectrophotometer at 25° C. Samples (50 μM peptide) were scanned from 260 to 200 nm (0.1 mm pathlength cell, 1.0 nm bandwidth, 0.5 nm resolution), in 10 mM Tris(hydroxymethyl)aminomethane buffer (Tris; pH=7.8). Eu(III) and Ca(II) were added as the chlorides. Aliquots of 10 mM stock metal solutions were added to a maximum of 10 equivalents.

The titration of free and metallated peptides with trifluorethanol (TFE) was followed by CD under similar conditions (1.0 cm pathlength cell). Aliquots of TFE were added to the buffered samples, which contained free peptide (25 μM P2 or P3), or 1:1 Eu-peptide (25 μM EuP2 or EuP3), in 5 mM Tris buffer (pH=7.8). Spectra were collected from 0 to 66% TFE.

Peptide helical content was calculated from molar ellipticity ([φ₂₂₂]) at 222 nm, based on the following formula: [φ₂₂]=(100×φ₂₂₂)/cn [symbol] , where φ₂₂₂ is millidegrees rotation at 222 nm, c is concentration of peptide in mM (corrected for dilution), n is the number of amino acid residues (33), and [symbol] is the pathlength in cm (Lehrman et al., 1990). Percent helicity was calculated assuming 100% [φ₂₂₂]=31,500 deg·cm²·dmol⁻¹ (Chen et al., 1972), and that only α-helical structure contributes to intensity at 222 nm.

¹H-NMR. The titration of P3 with Eu(III) was followed by ¹H-NMR. Spectra were collected on a 400 or 600. MHZ Brüker Spectrospin spectrometer at 298 K with a 5 mm BBO probe. Each spectrum was recorded with a sweep width of 8013 Hz (32 acquisitions, 32768 total points). Samples of P3 (0.96 mM) were prepared in a solution of 50 mM imidazole-d₄ (98%; Cambridge Isotopes) and 50 mM NaCl, pH=7.5. Aliquots of EuCl₃ (25 mM stock, pH=6.0) were added directly to the NMR tube and the sample thoroughly mixed. Each sample was allowed to equilibrate for at least ten minutes prior to data collection.

The effect of concentration on EuP3 solution structure was followed by ¹H-NMR (400 MHz). A sample of EuP3 (0.96 mM) was prepared in D₂O, 50 mM imidazole-d₄ buffer, 50 mM NaCl, pH=7.5. Aliquots of buffer were added, lowering the concentration of EuP3 to 0.33 mM. Spectra were recorded as above.

DNA Gel Shift Assays. The affinity of EuP3, EuP2, and each free peptide for supercoiled plasmid DNA was examined by agarose gel electrophoresis. Plasmid (pBR322, New England Biolabs, Inc.) was incubated for 15 minutes at room temperature with increasing concentrations of each ligand (5-25 μM), prior to the addition of loading dye. Each lane contained 1 μg plasmid (50 μM base pairs) in 10 mM Tris buffer at pH 8.0. Agarose gels (1% agarose in 1×TAE buffer (Tris.Acetate.EDTA) were run for approximately 2 hours at 70-80 volts, then stained with a 1 μg/mL ethidium bromide solution overnight. The gels were visualized under UV light and photographed with Polaroid 3000 ISO 667 film. The photographs were scanned, and DNA concentrations quantified with ImageQuant software (Molecular Dynamics). Control lanes containing only plasmid, buffer, and dye were treated in the same manner as other samples. No precipitation was observed in the samples. Similar results were found with pUC19 plasmid.

Metal Binding and Solution Structure. The binding affinity of P3 for Eu(III) was characterized by isothermal titration microcalorimetry. The dissociation constant for EuP3 was found to be 10±4 μM, from which the amount of bound and free Eu(III) in solution was calculated (Table 1). Though there is only one binding site per peptide, the binding behavior was not a simple two species process. EuP3 also dimerizes at higher concentrations K_{dim}≧80 μM. However, the second metal site in the dimer has low affinity (K_{d}>1 mM), so free Eu(III), EuP3 monomer, and a singly occupied dimer (EuP3), are the species present at concentrations below 100 μM.

TABLE 1
Rates of BNPP⁺ cleavage as a function of Eu-peptide at 37° C.

| Concentration | [EU_{free}] | [EuP3] | [EuP3₂] | Rates (K_{(obs)}) |
|---|---|---|---|---|
| (μM Eu/μM P3) | (calc; μM) | (calc; μM) | (calc; μM) | (s⁻¹ × 10⁷) |
| 10/10^{a} | 6.2 | 3.7 | 0.3 | 6.0 |
| 25/25^{a} | 11.6 | 11.8 | 1.8 | 7.3 |
| 25/50^{a} | 5.2 | 15.3 | 5.3 | 20.6 |
| 50/50^{b} | 18 | 27 | 5 | 17.5 ± 4 |
| 50/100^{b} | 8 | 28 | 14 | 11.5 ± 4 |
| 10/0^{b} | 10 | — | — | 5.2 ± 0.6 |
| 12/0^{b} | 12 | — | — | 5.7 ± 0.3 |
| 20/0^{b} | 20 | — | — | 19 ± 3 |
| Eu/P4a | [EU_{free}] | [EuP4a] | [EuP4a₂] | |
| 10/10^{a} | 4 | 6 | | 11.3 |
| 25/25^{a} | 7 | 18 | | 56.3 |
| 25/50^{a} | 2 | 22 | 1 | 62.0 |
| 50/50^{a} | 11 | 38 | 1 | 149.5 |
| 50/100^{a} | 2 | 44 | 4 | 212.0 |
| EuCl₂ | [EU_{free}] | — | — | |
| 10/0^{a} | 10 | — | — | 1.4 |
| 15/0^{a} | 15 | — | — | 2.0 |
| 20/0^{a} | 20 | — | — | 2.7 |
| Eu/EuP5L | [EU_{free}] | [EuP5L] | [EuP5L₂] | |
| 10/10^{b} | 7.2 | 2.8 | 0 | 19.5 ± 4 |
| 50/50^{b} | 23 | 22 | 5 | 30.8 ± 3 |

⁺BNPP concentration = 500 μM, pH = 7.7, 10 mM Tris buffer^{a} or 5 mM Tris buffer^{b}.
^{a}Calculated [EuP3], [EuP3₂], and [Eu_{free}] values are based on the measured dissociation constants for EuP3 (K_{d} = 10 μM; K_{dim} ≧ 80 μM) and EuP4a (K₁ = 3 μM: K_{dim} ≧ 300 μM).
^{b}Calculated [EuP3], [EuP3₂] and [EU_{free}] values are based on the measured dissociation constant for EuP3 (K_{d} = 10 μM). Calculated [EuP5L] and [Eu_{free}] values are based on an estimate of K_{d} = 20 μM. Error limits; K_{obs} = ±2%.

Results

Chimeric Design. Overlays of the HTH and EF-Hand motifs showed remarkable similarity in overall shape, and served as a basis for chimeric design (FIG. 2). The crystal structure coordinates of several homeodomain (engrailed, with and without co-crystallized DNA, Clark et al., 1994 and Tucker-Kellogg et al., 1995; and Antennapedia without, Frankel et al., 1998) and Ca-binding proteins (calmodulin, Chattopadhyaya et al., 1992; parvalbumin, Roquet et al., 1992; and calcineurin, Kissinger et al., 1995) were obtained from the Protein Data Bank, and overlayed using the protein visualization program SwissPDBViewer. The EF-Hand loop region was aligned such that the helix axes of each motif were colinear. These motifs consist of two helices at approximate right angles to one another, and as such, can be overlaid either parallel or antiparallel. The parallel orientation is required for correct sequence design. Generally, overlays showed similar helix orientations in 3-dimensions, though some deviation in α-α angle between various HTH or EF-Hand motifs resulted in a range of fits. The best fits (determined by inspection and RMS deviation of small helical sections) were used for further peptide sequence design. For example, the HTH of engrailed homeodomain (residues 27-56, 1ENH) was found to be particularly complementary in α-α angle to the third EF-Hand loop of calmodulin (residues 93-104, IOSA) and to the second EF-Hand loop of parvalbumin (residues 79-108, 5PAL, FIG. 2).

In order to maintain the same 3D orientation of α2 and α3, the last turn of α2 needed to be omitted when including the EF-loop. If just the four residues of the turn from the HTH were replaced, then α2 was displaced in space one turn (about 4 Å) in the N-terminal direction, destroying potential α2-α3 hydrophobic stabilization at the turn. Based on these observations, a peptide was designed and synthesized (FIG. 3). P3 comprises α2 and α3 of engrailed (T₂₇-L₃₄ and E₄₂-K₅₇) and the twelve-residue consensus EF-Hand Ca-binding loop (Falke et al., 1994). Three additional residue substitutions were made (X₍ₙ₎ denotes numbering scheme). The substitution of A₄₃-R₍₁₉₎, the residue which occurs in the related Antennapedia homeodomain sequence, incorporated an additional basic residue to strengthen electrostatic interactions with DNA. A second modification (Q₄₄-E₍₂₀₎) maintained the conserved Glu at the twelfth position of the EF-Hand loop, and the W₄₈-H₍₂₄₎ substitution was incorporated for ease of synthesis by Fmoc chemistry. Based on the parent crystal structures, sites of Ca(II) or Ln(III) binding are indicated by an x, sites of phosphate backbone contact with an o, and DNA base contacts with a * for representative P3. P3W (SEQ ID NO:7) has two of the three substitutions in P3. P3a (SEQ ID NO:10) has a truncated C-terminus relative to P3 and P3W and a I₄₆-Q₍₂₁₎ substitution.

P4 (SEQ ID NO:4) comprises α2 and α3 of Engrailed, minus the last turn(s) of α2 and the β-turn, and contains calmodulin loop I, P5 (SEQ ID NO:5) comprises α2 and α3 of Antennapedia and calmodulin loop III. The abbreviated 20-mer peptide P5L comprises the loop region of P5 (F₆→F₂₅). P4, P4a and P5 incorporate a greater fraction of the EF-Hand turn which likely improves the fold, as the native salt bridges (Arg-Glu) and hydrophobic contacts (Phe-Phe or Phe-Val for P4a) between the first turns of helix E and helix F are retained. CM1 (SEQ ID NO:6) is a loop modified Engrailed peptide.

A control peptide (P2) was also synthesized. This peptide included the same features as P3, but with the 12-residue consensus Ca-binding loop sequence reversed. P2 was predicted not to bind or fold effectively, allowing the comparison of positive and negative de novo design within synthetic peptides of similar size and construction. The helical regions of engrailed (α2 and α3) were incorporated as in P3, but with a difference in register, based in part on an anti-parallel structural alignment. No substitution for W₄₈ was made in P2 (W₍₂₆₎).

Circular Dichroism Titrations. To establish that these peptides have structure in solution, and whether this structure is influenced by metal binding, the induced helicity of the peptides was measured as a function of added metal. The CD spectra of free P2 and P3, and each peptide with La(III), Eu(III), and Ca(II) was investigated in Na₂HPO₄ and Tris.HCl buffers (25-50 μM peptide). The spectra had minima at 222 run, indicating some amount of α-helical structure (Saxena et al., 1971). For the control peptide (P2; 50 μM), the addition of metal does not appreciably increase secondary structure. The metal-saturated P3 spectrum is nearly identical for Eu(III) and La(III), but was not achieved at 100-fold excess Ca(II) (25 μM peptide). This is consistent with the expected lower binding affinity of the divalent ion. No further changes are seen in the 100-fold excess La(III) and Eu(III) spectra.

The secondary structure of P3 as a function of metal was followed by CD, calculating the % α-helicity from the molar ellipticity at 222 nm (100% helical=−31,500 deg cm² dmol⁻¹). With the addition of Eu(III) to P3, the helicity increases from 9 to 25%, showing enhanced structure correlated to metal-coordination. This increase in structure with added metal describes a curve which has an inflection point at approximately 1:1 Eu:P3, then continues to a 25% helical metal-saturated form. Metal binding affinity (K₁) can be estimated from the initial portion of the curve (0-50 μM) to be K₁ about 10 to 20 μM, in good agreement with the calorimetry data. A second structural change, correlating to the discrete back-to-back dimerization which has been well characterized for similar EF-Hand peptides systems occurs at K_{dim} about 80 μM (Shaw et al., 1991; Wójcik et al., 1997; Clarke et al., 1993; Shaw et al., 1990; Maurer et al., 1995). The addition of Ca(II) causes the same initial structural change (increasing to 14% helical content), but no further change in the metal saturated form. This may reflect weaker, less rigid binding by the 2⁺ versus 3⁺ ions, as described by Clark with similar isolated EF-Hand loops (Clark et al., 1993), or less tendency to dimerize with Ca(II) ions, as described by Wójcik et al. (1997).

Induced Secondary Structure. The nucleation of structure by metals can be indirectly studied by the observation of CD spectral changes with added trifluorethanol (TFE), an agent known to enhance α-helicity and β-hairpins in proteins by favoring internal protein hydrogen bonds over those of the solvent (Lu et al., 1997, Cammers-Goodwin et al., 1996; Starrs et al., 1992). TFE serves to stabilize local minima in structure. Thus, a helix induction curve (molar ellipticity versus % TFE) that has plateaus indicates something more than random helix formation, e.g., a preferred 2° or 3° structure within the protein, and can therefore illuminate differences in the inherent structural stability of various species.

The helix induction curve for free P2 and P3 peptides, and each with Eu(III) (25 μM Eu:peptide, 5 mM Tris buffer, pH=7.8) shows that all have plateaus in the curve, showing that they have inherent structural tendencies. The peptides alone initially have very similar behavior, likely because of the similarity in the α2, α3 regions derived from the homeodomain, which have a propensity to form helices. However, the Ca-binding loop of P3 resists further α-helix formation at higher TFE concentrations, since the loop has a preference for β-turn structure. P2, in contrast, has no defined loop, so a further linear increase in helicity is seen at greater than 40% TFE.

Upon the addition of metals, very different behavior is seen for the designed and control peptides. For P2, added Eu(III) decreases helicity (even at 0% TFE), inhibiting the tendency of the peptide to form helices. For P3, however, added Eu(III) significantly increases the α-helicity, reaching a plateau at an α-helical content similar to the metal saturated spectrum in water (at 50 μM peptide). As was the case for free P3, EuP3 resists further structural changes until a much higher percent TFE. The helix induction curve suggests that EuP3 has a tendency to adopt a defined structure with significant helical content, consistent with a native-like fold.

¹H-NMR. The 400 and 600 MHZ ¹H-NMR spectrum of P3 in D₂O in the presence and absence of equimolar Eu(III) shows changes in solution structure upon binding. The peaks in the spectrum of the apo-peptide are sharp and have little signal dispersion. The addition of EuCl₃ results in new resonances due to metal-bound peptide in slow-exchange with free P3. The new resonances are somewhat broadened relative to free P3 signals, but still well resolved. Appreciable mixing time between additions (up to 30 minutes) is required for full equilibration.

Several peaks characteristic of folded EF-Hands appear as a function of metal (Shaw et al., 1991; Shaw et al., 1990; Akke et al., 1991; Chen et al., 1998; Shaw et al., 1996). Particularly diagnostic are the upfield shifts of Ile γ CH₃ protons, to 0.6-0.7 ppm. The changes in the spectrum due to Eu-bound peptide predominantly occur within the first 0.5 equivalents of metal added, supporting the conclusion that the peptides dimerize to a singly-occupied EuP3₂ structure. This titration behavior is also observed with native EF-Hand peptides (Shaw et al., 1991; Shaw et al., 1990; Akke et al., 1991; Chen et al., 1998; Shaw et al., 1996), for which one metal ion is sufficient to nucleate the fold of two EF-Hand peptides. However, at the relatively high concentration of the NMR experiment (0.96 mM), a second lower affinity site can also be populated. Thus the spectrum continues to change with the titration of the second half-equivalent of metal, to give the fully occupied dimer form. As this solution is diluted, the low affinity site becomes unpopulated, and the spectrum approaches that of the singly-occupied dimer recorded with 0.5 equivalents of metal.

Both the binding affinity estimates from CD and microcalorimetry, and the stoichiometry of the EuP3 NMR titration support the conclusion that the spectral changes are due to dimerization, and not simple metal binding. The synthetic peptides thus mimic EF-Hand behavior, dimerizing first to a singly-occupied Eu(P3)₂ structure analogous to the native EF-Hand fold, then to the fully metallated dimer, Eu₂(P3)₂.

DNA Affinity. The interaction of these peptides with DNA was investigated by agarose gel electrophoresis assays of supercoiled plasmids. The interaction of ligand with supercoiled plasmid (type I) can be observed if run under conditions where the DNA-ligand interactions are not disturbed during electrophoresis. Supercoiled pBR322 was chosen as a standard plasmid, and was incubated for 15 minutes with EuCl₃, EuP3, EuP2, or free peptide prior to electrophoresis. This small, 4,361 bp plasmid contains three 5′-TAATT-3′ sequences, and eight 5′TAAT5′ sequences, partial target sequences for engrailed homeodomain. In the absence of 0.1 M EDTA (normally added prior to electrophoresis to quench hydrolytic cleavage assays) and with short incubation times to minimize metal-catalyzed cleavage, a gel shift can be observed. This gel shift and disappearance of type I plasmid occurs as a function of EuP3, but not EuCl₃ alone. Additionally, there is no shift due to EuP2, suggesting that the sequence, and thus structure, of the peptide is important for this effect. P3 has two additional positive residues relative to P2, which may enhance DNA interactions, and in fact, a gel shift is also observed for free P3, though at slightly higher concentrations.

From quantified concentration-dependent gel shift data, an estimate of the binding affinity of EuP3 can be made. Gels with increasing Eu-peptide concentrations (5 to 25 μM in each set) were quantified (ImageQuant; Molecular Simulations), and average band volumes were plotted versus concentration. The data indicate that the EuP3 binds DNA in the 20 μM range. No such shift is observed for EuP2.

Hydrolytic Phosphate Cleavage. Because the EF-Hand is physiologically strictly a structural motif, an isolated Ca-binding loop's ability to affect hydrolytic cleavage was addressed. The hydrolysis of bis-nitrophenylphosphate (BNPP) was followed spectrophotometrically under turnover conditions. The absorbance increase at 400 nm due to liberated 4-nitrophenolate was observed over the initial 10-14 hours of the reaction (<10% BNPP converted). No measurable hydrolysis of BNPP was observed in the absence of metal. Plots of absorbance vs. time were converted to concentration units (ε=18,500 M⁻¹ cm⁻¹) to give first order rate constants (Sardesai et al., 1994).

The synthetic metallopeptide catalyzes BNPP hydrolysis with rate constants on the order of k=10⁻⁶ sec⁻¹ (Table 1), comparable to other Ln catalysts (Morrow, 1994; Chappel et al., 1998). This represents a rate increase of approximately 10⁶ over uncatalyzed reactions, showing that the metal in the Ca-binding motif is indeed accessible enough to be hydrolytically active (at 37° C., pH=7, BNPP is hydrolyzed with an estimated rate of 6×10¹¹) (Chin et al., 1989). While EuP3 cleavage may not be as fast as free Eu(III) catalysis, catalysis by EuP5L apparently is. This may be due to a more flexible and open coordination environment in this abbreviated peptide. Notably, the concentrations of free Eu(III) calculated from the measured dissociation constants are not alone sufficient to explain the cutting rates.

Moreover, EuP3 catalyzes the cleavage of supercoiled, double-stranded DNA as well as model compounds. The conversion of supercoiled plasmid (type I) to open circular (type II), linear (type III), or smaller fragments was monitored by agarose gel electrophoresis. Because the synthetic peptides bind strongly to DNA, thus preventing the observation of products, the peptides were chelated prior to electrophoresis (Falke et al., 1994). At the point each reaction was quenched (0.1 M EDTA), a suspension of neutral, washed, cation resin (Amberlyst, 10-20 μL) was incubated with each sample for 30 minutes, spun down, and the supernatent loaded into wells. After 24 hours of reaction (incubated at 37° C.), the concentration-dependent formation of open circular plasmid was observed. Higher concentrations of EuP3 are less effective (slower), in keeping with the model BNPP system. Also of note is that 25 μM EuP3 in the presence of 225 μM excess metal has a similar effect to 25 μM EuP3 alone, suggesting that peptide bound to DNA blocks indiscriminate Eu cleavage. Over a 10-300 μM EuP3 gradient, nicking occurred from 10-150 μM, with the greatest amount of cleavage at 30 μM EuP3. EuP3 activity falls off with increasing concentration likely due to dimerization. Surprisingly, EuP4a activity does not.

Interestingly, the observed phosphate and DNA cleavage rates show an inverse dependence on catalyst concentration, correlating to the dimerization of two P3 or EuP3 moieties. This suggests that while a monomeric EF-Hand is catalytically active, the metal ions in supersecondary native-like dimer structures are not accessible for catalysis. Thus, HTH/EF-hand chimeras bind metals, have metal-dependent solution structure, and interact with and cleave DNA.

Discussion

It is of great interest to generate artificial repressors to target sequences of choice, not only for the biochemical utility of such agents, but for the pharmaceutical impact of drugs which could target a single promoter region on the genome. Toward this end, an isolated peptide motif which binds Eu(III) and Ca(II), has enhanced solution structure as a function of metal, and binds DNA, in analogy to its unrelated parent domains was prepared. The chimeric approach to the design of selective DNA binding units employs the same highly specific, yet flexible protein-DNA interactions which cells themselves use to regulate their growth.

Based on their metal-binding and solution behavior, the synthetic peptides can be classified as true EF-Hand derivatives. Isolated EF-Hand peptides have been shown to have significant affinity for lanthanides, commonly ranging from K_{d}=10 to 50 μM depending on loop sequence (Dadlez et al., 1991; Goch, 1999; Siedlecka et al., 1999). This Ln(III) affinity is only slightly lower than that of typical EF-Hands in the context of proteins (K_{d}=6 nM to 3 μM) (Bruno et al., 1992; Burroughs et al., 1994; Drake et al., 1996), suggesting that this motif is inherently well-organized into an Efimov-type α-α turn. The CD titration showed that the designed peptide P3 folds as Eu(III) is incorporated, with K_{d}<20 μM, similar to native EF-Hand peptides. The control peptide P2, however, does not fold in the presence of Eu(III). Instead the helical content remains unchanged or even decreases (depending on peptide concentration) as a function of added metal. This is expected for random metal binding to various acidic sidechains; rather than nucleating helix formation by bringing together favorable hydrophobic and electrostatic residues as a single metal-binding site is organized, random binding to multiple weak sites would disfavor defined secondary structure.

The CD results for Eu(III) binding to P3 show a further equilibrium event which we assign to dimerization; both the metal-binding (K_{d}) and dimerization (K_{dim}) equilibria are evident in an isothermal microcalorimetry study as well. The helical content increases to a maximum value of 25%, both with excess Eu(III) and with the stabilization afforded by TFE solvent. This behavior is consistent with EuP3 adopting a defined rather than random structure in solution, in which metal-binding organizes the central loop region, and nucleates helix formation at either terminus. The calcium adduct, in contrast, reaches only 14% helicity in the metal-saturated form. This helicity difference may be due to a disparity in Ln(III)/Ca(II) dimerization behavior, as was observed for an abbreviated 13-mer EF-Hand loop peptide (Wójcik et al., 1997). The higher rigidity of the loop structure in the presence of the trivalent metals helps to explain why Ln(III) ions, but not Ca(II) ions, induce dimerization in the P3 peptide as well.

Part of the affinity of native EF-Hands for Ca(II) is due to the propensity of these motifs to occur in pairs within proteins (Falke et al., 1994). This tendency derives from the complementarity of the helix-loop-helix surfaces, and the hydrogen bonding of adjacent loops as short anti-parallel β-strands. Isolated EF-Hands peptides also dimerize in solution, forming back-to-back native-like folds (Maurer et al., 1995; Shaw et al., 1996; Monera et al., 1992). A metallated loop becomes a structural template for the folding of a second, apo-peptide into a back-to-back pair (MP•P). The metal ion does not bridge two EF-Hand motifs, but the dimerization is instead due to hydrophobic and β-sheet interfaces between strands. Thus, one equivalent of metal ion (M) organizes two equivalents of peptide (P). The second, lower affinity site (about 1 mM) is not populated until higher metal concentrations are reached, giving the full dimer (MP•PM) (Shaw et al., 1991). For native EF-Hand sequences, a monomeric intermediate (MP) is not even observed, as they instead fold directly to a MP•P form (Shaw et al., 1991; Shaw et al., 1990). As a result of the tertiary interactions, dimerization of the EF-Hand motif is favored even with truncated loops (Wójcik et al., 1997).

In addition to the CD titration results, further evidence for dimer formation and insight into the structure of the metallopeptides comes from NMR. The conformational changes in P3 with metal binding were followed by ¹H-NMR spectroscopy. The appearance and increase of new peaks upon the addition of Eu(III) are consistent with slow-exchange kinetics of metal-binding and dimerization. However, there are no well-defined peaks outside the diamagnetic window to indicate a close and rigidly constrained proximity to the paramagnetic ion. This observation is consistent with greater flexibility of the metal-binding region in the synthetic peptide, relative to native EF-Hands. This local flexibility would further exchange-broaden peaks arising from residues in close proximity to the paramagnetic metal and render them unobservable under these conditions.

Most changes in the P3 spectrum occur within the first 0.5 equivalent of added Eu(III), supporting the conclusion that these peptides dimerize to the same singly-occupied structure (Eu(P3)₂=MP•PM) as native EF-Hand peptides. One metal ion structurally organizes two peptides. The few additional changes which occur between 0.5 and 1 equivalent Eu(III), reflect binding of a second metal, to give the fully occupied dimer (Eu₂(P3)₂=MP•PM). This conclusion is supported by dilution studies, in which lowering the EuP3 concentration from 0.96 mM to 0.33 mM results in a spectrum qualitatively similar to that of 0.5:1 Eu:P3, including the disappearance of certain peaks. Over this dilution range, the first metal site remains fully occupied (K_{d}≦20 μM), and the monomeric EuP3 is less favored than a singly occupied dimer form (K_{dim}=80 μM). However, at the highest concentrations (approaching 1 mM Eu:P3), the lower affinity metal site must also become populated (K_{d2} about 1 mM), and this is reflected in the NMR spectral changes.

The synthetic peptide mimics its EF-Hand parent in its affinity for Eu(III) and Ca(II), and its tendency to dimerize. The legacy of the synthetic peptide's other parent motif is the DNA-binding affinity of the homeodomains. Agarose gel shift assays of EuP3 shows that the metallopeptide exhibits DNA-binding affinity in the micromolar regime. The DNA-binding is predominantly due to electrostatic interactions, as evidenced by the affinity of free P3. At pH=7, P3 has 10 positively charged residues, and three of the seven acidic residues (Asp, Glu) are charge-balanced by Eu(III) binding. Though P2 has two fewer basic residues, metallated P2 should still have greater total positive charge than apo-P3 (at pH=7, +4 versus +3, respectively). Thus, by electrostatic attraction alone, EuP2 should have greater affinity for DNA than free P3. However, neither P2 nor EuP2 has appreciable DNA affinity over the concentration range studied (5-25 μM). This suggests that the inherent tendency to fold evidenced by EuP3 (but not EuP2) plays some role in interactions with B-form DNA. This is expected if the synthetic peptide associates with the DNA major groove, as do native HTH domains.

Thus, a chimeric peptide motif can be prepared from two unrelated but topologically equivalent parent structures, while retaining the functions and features of the parent domains. The exemplified synthetic peptide binds Ca(II) and Eu(III) in a manner analogous to native EF-Hands, has solution structure and behavior consistent with a defined, helical fold, and exhibits significant affinity for supercoiled DNA as do HTH-containing repressors.

### EXAMPLE 2

Genetic mutations causing cancer and other diseases could be suppressed if target sequences could be selectively cleaved. Hydrolytic cleavage of DNA by Lewis-acid metal ions occurs with limited sequence discrimination, however, proteins are capable of selectively recognizing given duplex DNA sequences. Transcription factors and repressor proteins containing the helix-turn-helix motif (HTH) bind DNA tightly through a series of positively charged residues, which orient the recognition helix within the major groove of DNA. Sidechains of this helix contact and complement the base pair edges, resulting in the recognition of up to 18 base pairs for homodimeric transcription factors such as cro, lambda, or Trp repressors. As discussed above, the supersecondary structure of the HTH and the EF-hand, a calcium binding motif, are superimposable. This similarity can be exploited to design small synthetic peptides that bind lanthanides, retain the HTH fold and cleave DNA. The sequence specificity of two synthetic peptides was examined.

Metallopeptide Design. The α-α corner is a remarkably common structural motif (Efimov, 1997), stabilized by conserved hydrophobic interactions along the inner surfaces of two intersecting helices. The 33-mer peptide P3W was designed based on overlaid crystal structures of the α-α corner motifs from calmodulin (IOSA) and the engrailed homeodomain (1ENH). P3W comprises helices α2 and α3 of engrailed, with the turn region replaced by the consensus Ca-binding EF-hand loop. Structures were aligned using the freeware program SwissPDBViewer. Peptides (>95% pure) were obtained from New England Peptide (P3W) or the Caltech Peptide Synthesis Facility (P3). The design of peptide P3 included a Trp₂₄ to His₂₄ substitution for ease of synthesis. However, the native α3 sequence in P3W preserves the hydrophobic core of the HTH motif and results in a more rigid, well-structured metallopeptide. Additionally, important hydrophobic interactions known to stabilize EF-hand structures (Falke et al., 1994) were retained at positions −1/+13 (L₈-I₂₁) and −1/+16 (L₈-W₂₄), though the residues derive from the engrailed sequence (numbers are relative to +1, the first residue of the Ca-binding loop, or D₉ for peptide P3W).

Solution Structure and Lanthanide Binding. The designed peptides P3 and P3W bind lanthanide ions via the consensus EF-hand site and fold to a flexible but native-like structure, as demonstrated by CD titrations, fluorescence titrations, and NMR spectroscopy. P3W, in contrast to P3, has significant solution structure even as the free peptide (FIG. 3C), and folds at lower metal concentrations. Only one equivalent of Eu(III), La(III), or Ce(IV) results in an identical folded P3W spectrum (50 μM peptide), whereas saturating amounts of metal are required to promote a well-organized structure for the more flexible peptide P3. An estimate of secondary structure based on Compton fitting of the CD data (Olis software) indicates that the 1:1 LnP3W metallopeptides comprise percent α-helix, β-strand, and random coil secondary structure values which are similar to those predicted from the crystal structure of a single EF-hand of calmodulin. Importantly, an NMR solution structure model based on the chemical shift index method and NOE restraints showed that the LaP3W complex retains the helix-loop-helix structure of the parent motifs.

The binding affinity of P3W for lanthanides was determined by fluorescence spectroscopy, following the intensity of Trp₂₄. Because the peptide folds as a function of metal, changing the solvation environment of the Trp residue and bringing it into proximity of the metal, a non-linear least squares fit of the intensity data to a 1:1 association model gave Kd for each metallopeptide. Further equilibria, such as dimerization of the metallopeptides, did not improve the fit.

Sequence Selective DNA Cleavage. The Eu(III) and Ce(IV) complexes of P3W catalyze the hydrolysis of supercoiled DNA, producing primarily single cuts (open circular product) and demonstrating that an exposed EF-hand is catalytically competent. This cleavage is not dependent on the strain inherent in supercoiled plasmid, as cleavage of linearized ³²P-labeled DNA oligonucleotides was observed as well. Labeled fragments were incubated with Ce(NH₃)₆(NO₃)₄ and varying concentrations of peptides P3 or P3W at 37° C. overnight and analyzed by acrylamide gel electrophoresis. Cleavage was concentration dependent, and distinct between metallopeptides.

An important observation is the regioselectivity of cleavage by the metallopeptide. Hydrolysis by free Ce(IV) ion (“free metal ions” are ions not complexed by peptide, though they exist as aqua/hydroxide Tris-stabilized species under these conditions) is known to produce both 3′- and 5′-phosphate termini, as there is no stereoselectivity in the hydrolysis mechanism. However, in the presence of chimeric peptide, the double bands observed in the free Ce(IV) lanes are repressed, even when excess free metal is present. The resulting products comigrate with the Maxam-Gilbert sequencing lanes, which indicates the synthetic peptide is generating 3′-OPO₃ termini exclusively. As the retained phosphate is the larger fragment at each base step, this result does not support a single product arising from further cleavage masking the 3′-OH product.

In addition to regioselectivity, the synthetic peptides direct sequence recognition. At low peptide concentrations, a non-random pattern of cleavage is observed, with greater sequence discrimination for the well-folded CeP3W than for CeP3. This cleavage pattern suggests preferential binding to some sites over others. However, as the concentration of peptide increases, the metallopeptide populates lower affinity sites, eventually binding to any DNA sequence. At concentrations greater than shown, strong random binding results in aggregation to the point of precipitation. The metallopeptide CeP3W preferentially cleaves at sites indicated by the hashed arrows. On this particular fragment, the sites for which there is the greatest affinity are 5′-TRARC-3′ sites. This level of sequence selectivity is clearly lower than that observed for homeodomain proteins or other HTH-containing transcription factors, but is remarkable for a peptide of this size. Further, P3W enter cells in a metal-dependent manner.

This work illustrates how a robust fold can be redesigned to incorporate reactivity into a recognition motif. By retaining the HTH structure, the chimeric metallopeptide is capable of sequence discrimination, despite its small size.

### EXAMPLE 3

Methods

P3W peptide (TERRRQQLDKDGDGTIDEREIKIWFQNKRAKIK, Gd-binding EF-hand site is shaded) was synthesized and purified (>95% by HPLC) by the Caltech Peptide Synthesis Facility, Pasadena, Calif. The gadolinium complex was prepared by dissolving P3W in 50 mM HEPES buffer, pH 7.4, and adding 0.8 equivalents Gd(NO₃)₃ to give a 500 μM stock solution. Peptide concentration was estimated by absorbance (ε₂₈₀=7290 M⁻¹cm⁻¹); the absence of uncomplexed Gd³⁺ was confirmed by colorometric analysis with xylenol orange. All Gd concentrations were determined by ICP-MS (Agilent 7500). Relaxation rates were determined at 20 and 60 MHz, 37° C. using Broker NMS 120 and Brtiker mg60 Minispec spectrometers, respectively. NMRD profiles were recorded on a Koenig-Brown field cycling relaxometer; phantoms were imaged on a GE 1.5T clinical imager.

Results

If a chelating moiety is incorporated within a targeting group, internal motion and rotation flexibility may be reduced and greater relaxivities may be achieved upon receptor binding. Peptide-based chelators are an attractive platform for incorporating a Gd site into a biological recognition unit. For instance, substitution of Gd into Ca binding proteins (O₈ or O₉ donor set) can yield very high relaxivities (Lauffer, 1987). A peptide based Gd-chelator mimicking a Ca binding protein thus offers the potential of high relaxivity and a means to exploit protein structure-function relationships in recognition. Moreover, solid phase peptide synthesis offers great flexibility in modifying and incorporating targeting motifs.

A helix-turn-helix (HTH) chimera, a metallopeptide based on the incorporation of EF-hand Ca-binding loop into the DNA-binding HTH motif (Kim et al., 2001; Welch et al., 2003), was investigated as a putative DNA sensing contrast agent. The peptide chimera P3W is based on the structurally similar α-α corner motifs of two unrelated proteins: engrailed homeodomain, a DNA-binding transcription factor, and calmodulin, a Ca-binding signaling protein (Welch et al., 2003). Previous studies have shown that P3W folds upon binding one equivalent of trivalent lanthanide ions to the EF-hand Ca-binding site (Welch et al., 2001), binds DNA (Welch et al., 2001, and enters cells in the metallated form (data not shown). Circular dichroism and NMR studies of various Ln(III)P3W metallopeptides (Eu, Yb, and La) determined that the folded structure of the metallopeptide is analogous to the parental α-α corner motifs (Welch et al., 2003) (50 μM apo-P3W and GdP3W in 50 mM HEPES, pH 7.4. Estimates of helicity based on [Θ□₂₂₂ suggest that apo-P3W is about 18% helical and GdP3W is about 32% helical). The CD spectrum of GdP3W indicated that the gadolinium-peptide was similarly well-folded in solution, and the affinity for Gd(III) was comparable to that of Eu(III) (log K_{EuP}=5.21±0.3; log K_{GdP}=5.19±0.4, pH 7.8). The EuP3W complex was found by Eu-luminescence spectroscopy to have two inner sphere water molecules (q is about 2.0) both in the presence and absence of DNA, suggesting that q=2 GdP3W as well.

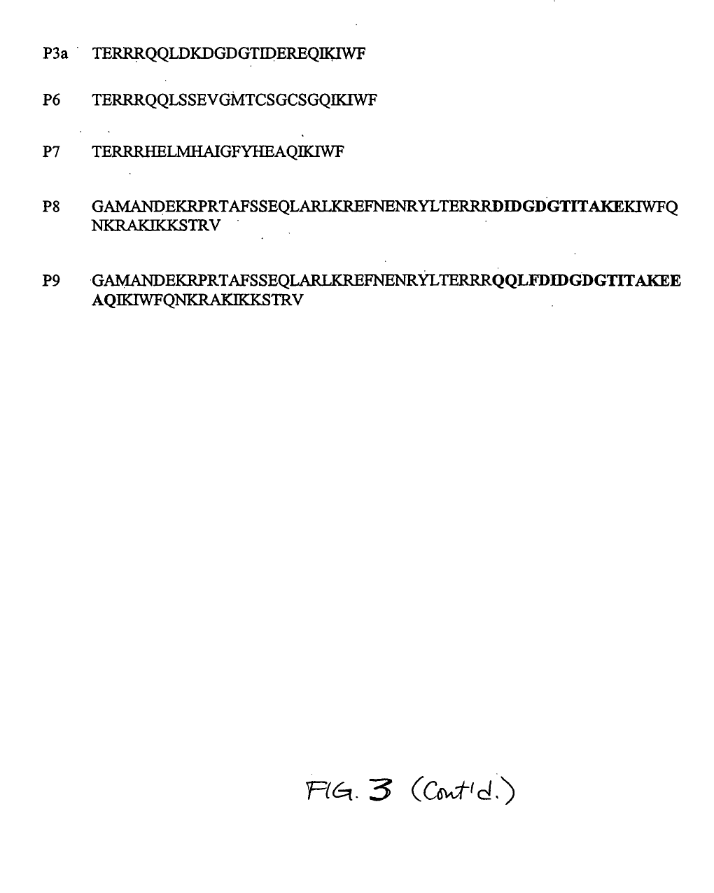

The relaxivity of GdP3W was determined at 20 and 60 MHz (37° C.). The GdP3W metallopeptide was found to be a very efficient catalyst of water proton relaxation (Table 2), with r₁=21.2 mM⁻¹s⁻¹ at 60 MHz, the most common clinical imaging frequency. This relaxivity is significantly higher (6-fold) than commercial agents, e.g., GdDTPA (Table 2). In the presence of one equivalent of DNA, the relaxivity at 60 MHz increased by 100% to 42.4 mM⁻¹s⁻¹. The increase in r₁ upon DNA binding was expected, but the magnitude and unusual field dependence (FIG. 6) of the relaxivity was surprising. Unlike most slow tumbling complexes reported, the relaxivity at 60 MHz was significantly greater than at 20 MHz, notable because most clinical MRI scanners operate at 1.5 tesla (about 65 MHz). It is clear from FIG. 6 that the relaxivity of the Gd-peptide-DNA complex is peaking near 60 MHz. This is likely a consequence of a difference in electronic relaxation imparted by the all oxygen donor set, suggesting that this ligand environment may be a useful design feature in contrast agents. With the exception of the hydroxypyridonate complexes developed by Raymond and coworkers (O₈ donor set) (Xu et al., 1995; Sunderland et al., 2001; Hajela et al., 2000; Cohen et al., 2000), Gd complexes proposed as contrast agents typically have mixed N,O donor sets, and are usually based on polyaminocarboxylato ligands. The relaxivity of GdP3W is much greater than the q=2 Gd-TREN-1-Me=3,2-HOPO and related complexes (about 6-11 mM⁻¹s⁻¹ at 20 MHz and 37° C.), presumably because of the embedded design of GdP3W which limits the internal motion of the complex. The relaxivity at 60 MHz is one of the higher relaxivities reported at this frequency, including other RIME type agents. For instance, it is approximately double the relaxivity of the albumin targeted contrast agent MS-325 (23 mM⁻¹s⁻¹ at 60 MHz) (Caravan et al., 2002).

TABLE 2
Relaxivities (mM⁻¹s⁻¹) of GdP3W and GdDTPA (37° C., 50 mM
HEPES, pH 7.4) at 20 and 60 MHz in the presence and absence
of one equivalent DNA (14-mer d.s. DNA: self-complementary 5′-
GAGCTAATTAGCTC-3′; SEQ ID NO: 13).

| | |
|---|---|
| 20 MHz | 60 MHz |

| Contrast Agent | r₁ | r₁ + DNA | r₁ | r₁ + DNA |
|---|---|---|---|---|
| GdP3W | 16.2 | 29.6 | 21.2 | 42.4 |
| GdDTPA | 4.6 | 4.9 | 3.5 | 3.5 |

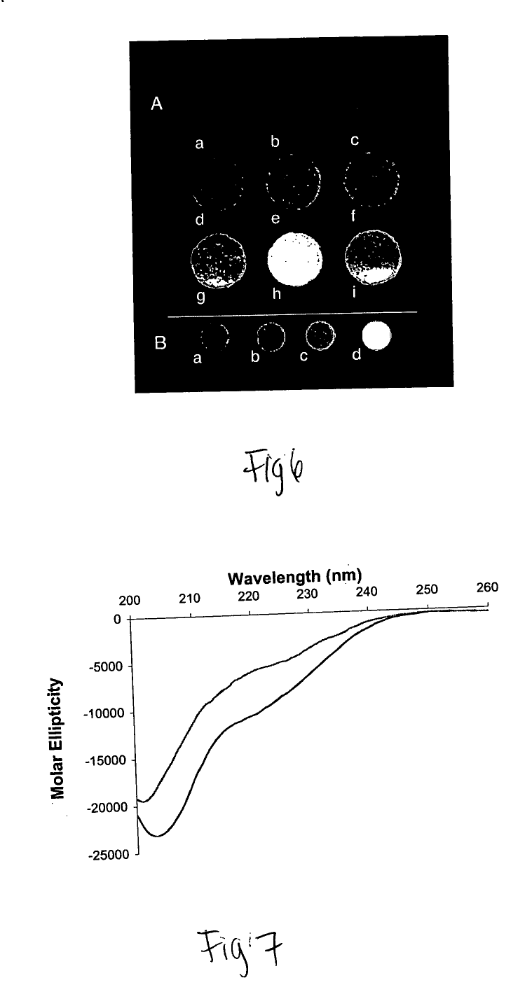

High relaxivity is an important feature in the development of new contrast agents. Since MRI contrast agents are detected indirectly by their influence on water relaxation rates, relatively high concentrations of contrast agent are required. The benefit of a high relaxivity agent such as GdP3W is qualitatively shown in FIG. 7A. T₁-weighted imaging (short T₁ giving positive contrast) at 1.5 T shows that at a fixed gadolinium concentration of 50 μM, the contrast between GdDTPA and buffer is difficult to discern (FIG. 7B). However, GdP3W is significantly brighter than the clinical agent, and when “switched on” by the presence of DNA, the signal is brighter still. The DNA “switch” concept is shown by the addition of 1 equivalent of DNA to the GdP3W sample, resulting in a signal increase relative to GdP3W upon target binding (image h versus image g). This emphasizes the importance of high relaxivity: if the target concentration is low, then it will be undetectable unless the relaxivity is high.

An additional study showed that the differences in relaxivity were borne out by the images: a 8.6 μM GdP3W+DNA phantom (image f) was isointense with 115 μM GdDTPA (image d) demonstrating that the same contrast can be achieved with 12 times less gadolinium. Addition of DNA did not increase the signal of the GdDTPA sample (images d and e). Moreover, DNA alone or P3W alone gave no signal enhancement. The electrostatic binding interaction of DNA and GdP3W could be “salted out” by a large excess of NaCl, which caused a decrease in r₁ back to that of GdP3W alone (demonstrated in phantoms or by measuring T₁ as a function of NaCl added). This demonstrates that the increase in GdP3W signal in the presence of DNA is truly due to reversible DNA-binding by the metallopeptide.

This study shows that a Gd(O)₈ site in the context of a peptide-based DNA-targeting moiety can enhance T₁ contrast dramatically compared to commercial agents, and this contrast can be modulated by DNA-binding. Both the larger size (MWt>4000 Da), causing a slower tumbling rate, and the two inner-sphere water molecules, contribute to the increased relaxivity of GdP3W relative to GdDTPA. The dramatic increase in relaxivity upon target binding also suggests that incorporating rather than appending the Gd site to the targeting moiety is an effective approach to reducing rotational flexibility at the metal site. This unique dependence with increased sensitivity at a higher fields may be a general phenomenon for EF-hand-based metallopeptides, perhaps as a consequence of the unusual (for contrast agents) Gd(O)₈ coordination set.

Thus, chimeric HTH metallopeptides represent a new class of bifunctional targeted MRI agents with an embedded metal binding site, activated by and reporting on DNA-binding.

References

Aime et al., Angew. Chem. Int. Ed. Engl., 41, 1017 (2002).
Aime et al., Biopolymers, 41, 1017 (2002).
Akke et al., J. Mol. Biol., 66, 419 (2003).
Almquist et al., J. Med. Chem., 23, 1392 (1980).
Bavaay and Merrifield, The Peptides (E. Gross and F. Meienhofer eds., Academic Press, 1980).
Bradford, Anal. Biochem., 72, 248 (1976).
Brennan et al., Trends Biochem. Sci., 14, 286 (1989).
Bruno et al., Biochemistry, 31, 7016 (1992).
Burley et al., Curr. Opin. in Struct. Biol., 4, 3 (1994).
Burroughs et al., Biochemistry, 33, 10428 (1994).
Cammers-Goodwin et al., J. Am. Chem. Soc., 118, 3092 (1996).
Caravan et al., Chem. Soc., 99, 2293 (1999).
Caravan et al., J. Am. Chem. Soc., 124, 3152 (2002).
Celio et al., Guidebook to the Calcium Binding Proteins (Sambrook and Tooze Publication at Oxford Univ. Press 1996).
Chappel et al., Inorg Chem., 37, 3989-3998 (1998).
Chattopadhyaya et al., J. Mol. Biol., 228, 1177 (1992).
Chen et al., Biochemistry, 11, 4120 (1972).
Chen et al., J. Biol. Chem., 273, 13537 (1998).
Chin et al., J. Am. Chem. Soc., 111, 186 (1989).
Clark et al., Analy. Biochem., 213, 296 (1993).
Clarke et al., Protein Sci., 3, 1779 (1994).
Clark-Lewis et al., Meth. Enzymol., 287, 233 (1997).
Cohen et al., Inorg. Chem., 39, 5747 (2000).
Coruh et al., Biochemistry, 31, 7970 (1994).
Dadlez et al., FEBS Lett., 282, 143 (1991).
Dayhoff, Atlas of Protein Sequence and Structure, volume 5, National Biomedical Research Foundation, pp. 101-110, and Supplement 2 to this volume, pp. 1-10 (1972).
DeJong et al., J. Nucl. Med., 40, 2081 (1999).
Drake et al., Biochemistry, 35, 6697 (1996).
Efimov, FEBS Lett., 166, 33 (1984).
Efimov, FEBS Lett., 355, 213 (1994).
Efimov, FEBS Lett., 391, 167 (1996).
Efimov, Proteins: Structure, Function and Genetics, 28, 241 (1997).
Evans et al., J. Med. Chem., 30, 1229 (1987).
Falke et al., Quart. Rev. Biophys., 27, 219 (1994).
Falke et al., Biochemistry, 30, 8690 (1991).
Fauchere, Adv. Drug Res., 15, 29 (1986).
Flacke et al., Circulation, 104:1280 (2001).
Fraenkel et al., Nat. Struct. Biol., 5, 692 (1998).
Freemont et al., Biochem. J., 1 (1991).
Gehring et al., Annu. Rev. Biochem., 63, 487 (1994).
Goch, Acta. Biochem. Pol., 46, 673 (1999).
Guex et al., Electrophoresis, 18, 2714 (1997).
Hajela et al., J. Am. Chem. So., 122, 11228 (2000).
Hann, M. M., J. Chem. Soc. Perkin Trans., I, 307 (1982).
Holladay et al., Tetrahedron Lett., 24, 4401 (1983).
Hruby, Life Sci., 31, 189 (1982).
Hudson et al., Int. J. Pept. Prot. Res., 14, 177 (1979).
Jennings-White et al., Tetrahedron Lett., 23, 2533 (1982).
Kim et al., J. Biol. Inorg. Chem., 6, 173 (2001).
Kissinger et al., Nature, 378, 641 (1995).
Kissinger et al., Cell, 63, 579 (1990).
Kornberg, J. Biol. Chem., 268, 26813 (1993).
Lauffer, Chem. Rev., 87:901 (1987).
Lauffer, Magn. Reson. Med., 22, 339 (1991).
Laughon, Biochemistry 30, 11357 (1991).
Lehrman et al., Biochemistry, 29, 5590 (1990).
Louie et al., Nature Biotech., 18, 321 (2000).
Luo et al., Biochemistry, 36, 8413 (1997).
Maurer et al., J. Mol. Biol., 253, 347 (1995).
Meienhofer, Hormonal Proteins and Peptides (C. H. Li ed. Academic Press, 1973).
Merrifield, J. Am. Chem. Soc., 85, 2149 (1963).
Moats et al., Angew. Chem. Int. Ed. Engl., 36, 726 (1997).
Monera et al., Protein Sci., 1, 945 (1992).
Morley, Trends. Pharm. Sci., 463 (1980).
Morrow et al., Models in Inorganic Chemistry, 9, 41 (1994).
Needleman and Wunsch, J. Mol. Biol., 48, 443 (1970).
Nivorozhkin et al., Angew. Chem., Int. Ed. Engl., 40, 2903 (2001).
Pabo et al., Annu. Rev. Biochem., 53, 293 (1984).
Patikoglou et al., Annu. Rev. Biophys. Biomol. Struct., 26, 289 (1997).
Fundamental Immunology (Paul, W. E. ed., Raven Press 1984).
Pearson and Lipman, Proc. Natl. Acad. Sci., 85, 2444 (1988).
Pomerantz et al., Science 267, 93 (1995).
Prochiantz, Ann. NY Acad. Sci., 886, 172 (1999).
Richardson et al., Biophys. J., 63, 1186 (1992).
Roquet et al., J. Mol. Biol., 223, 705 (1992).
Sambrook et al., In Molecular Cloning: A Laboratory Manual (1989).
Saxena et al., Proc. Nat. Acad. Sci., 68, 969 (1971).
Shaw et al., J. Am. Chem. Soc., 113, 5557 (1991).
Shaw et al., Science, 249, 280 (1990).
Shaw et al., Biochemistry, 35, 7429 (1996).
Siedlecka et al., Proc. Natl. Acad. Sci., 96, 903 (1999).
Simmons et al., Science, 276, 276 (1997).
Smith and Waterman Adv. Appl. Math., 2, 482 (1981).
Sovoboda et al., J. Biochem. Biophys. Acta, 1055, 207 (1990).
Spatola, Vega Data, 1, “Peptide Backbone Modifications” (1983). Spatola, Chemistry and Biochemistry of Amino Acids, Peptides, and Proteins (B.
Weinstein, ed. Marcel Dekker 1983).
Spatola et al., Life Sci., 38, 1243 (1986).
Starrs et al., Biopolymers, 32, 1695 (1992).
Stewart et al., Solid Phase Peptide Synthesis (W. H. Freeman Co. 1969).
Sunderland et al., Inorg. Chem., 40, 6746 (2001).
Treisman et al., BioEssays, 14, 145 (1992).
Tucker-Kellogg et al., Structure (London), 5, 1047 (1997).
Veber et al., T.I.N.S., 392 (1985).
Weissleder et al., Radiology, 219, 316 (2001).
Welch et al., Proc. Nat. Acad. Sci., USA, 100, 3725 (2003).
Wójcik et al., Biochemistry, 36, 680 (1997).
Wunderbaldinger et al., Acad. Radiol., 9, S304 (2002).
Xu et al., J. Am. Chem. Soc., 117, 7245 (1995).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A magnetic resonance imaging contrast agent comprising a synthetic peptide or polypeptide comprising at least two domains that together specifically bind nucleic acid and one or more domains that specifically bind a paramagnetic metal, wherein one of the domains that specifically binds the paramagnetic metal is between the domains that bind nucleic acid.

2. The magnetic resonance imaging contrast agent of claim 1 wherein the peptide or polypeptide comprises a consensus EF-Hand sequence.

3. The magnetic resonance imaging contrast agent of claim 1 wherein the domains that specifically bind nucleic acid are from a transcription factor.

4. The magnetic resonance imaging contrast agent of claim 3 wherein the transcription factor is engrailed.

5. The magnetic resonance imaging contrast agent of claim 3 wherein the transcription factor comprises a helix-turn-helix domain.

6. The magnetic resonance imaging contrast agent of claim 1 wherein the domains that specifically bind nucleic acid comprise a helix-turn-helix motif, a relaxed helix-turn-helix motif, a winged helix-turn-helix motif, a helix-loop-strand motif, or a hormone receptor motif.

7. The magnetic resonance imaging contrast agent of claim 6 that comprises alpha-helices 2 and 3 of a helix-turn-helix motif.

8. The magnetic resonance imaging contrast agent of claim 1 that binds dsDNA, dsRNA, ssDNA, ssRNA, A-DNA, B-DNA, or Z-DNA.

9. The magnetic resonance imaging contrast agent of claim 1 wherein the domains that specifically bind nucleic acid are from a homeodomain.

10. The magnetic resonance imaging contrast agent of claim 1 wherein the peptide or polypeptide binds calcium.

11. The magnetic resonance imaging contrast agent of claim 1 wherein the peptide or polypeptide comprises the amino acid sequence TERRRQQLDKDGDGTIDEREIKIWFQNKRAKIK (SEQ ID NO: 1), TERRRFDKDGNGYISAAELRHVKIWFQNKRAKIK (SEQ ID NO:3), TERRRFRVFDKDGNGYISAAEKIWFQNKRAKIK (SEQ ID NO:4), TRRRRFLSFDKDGDGTITTKEEVWFQNRRMKWK (SEQ ID NO:5) TERRRQQLDKDGDGTIDEREIKIWFQNKRAKIK (SEQ ID NO:7), TERRRQQLDKDGDGTIDEREQIKIWF (SEQ ID NO:10), DEKRPRTAFSGEQLARLKREFNENRYLTERRRLRVFDKDGNGFISAAEKI WFQNKRAKIKKST (SEQ ID NO:6), TERRRQQLSSEVGMTCSGCSGQIKIWF (SEQ ID NO:8), TERRRHELMHAIGFYHEAQIKIWF (SEQ ID NO:9) TERRRDIDGDGTITAKEKIWF (SEQ ID NO:13), TERRRQQLFDIDGDGTITAKEEAQIKIWF (SEQ ID NO:14), TERRRQQLSHGGGWGEAQIKIWFQNKRA (SEQ ID NO:15), or a portion thereof which specifically binds nucleic acid and specifically binds the paramagnetic metal.

12. The magnetic resonance imaging contrast agent of claim 1 wherein the peptide or polypeptide comprises at least 20 residues.

13. The magnetic resonance imaging contrast agent of claim 1 wherein the peptide or polypeptide binds Gd(III), Fe(III), Mn(II), Mn(III), Dy(III), Yt(III), or Cr(III).

14. The magnetic resonance imaging contrast agent of claim 1 which is at least 99 wt.% pure.

15. The magnetic resonance imaging contrast agent of claim 1 wherein the peptide or polypeptide binds a lanthanide.

16. The magnetic resonance imaging contrast agent of claim 1 which is suitable for intravenous delivery to mammalian tissue.

17. The magnetic resonance imaging contrast agent of claim 1 wherein the peptide or polypeptide is synthetically produced, or is semi-synthetically produced.

18. The magnetic resonance imaging contrast agent of claim 1 having a molecular weight of greater than about 4,000 Da.

19. The magnetic resonance imaging contrast agent of claim 1 further comprising a protein transport domain.

20. The magnetic resonance imaging contrast agent of claim 1 further comprising a metal.

21. The magnetic resonance imaging contrast agent of claim 1 further comprising a lanthanide.

22. The magnetic resonance imaging contrast agent of claim 1 further comprising Gd(III), Fe(III), Mn(II), Mn(III), Dy(III), Yt(III), or Cr(III).

23. A pharmaceutical composition comprising a pharmaceutically acceptable carrier and a magnetic resonance imaging contrast agent comprising a synthetic peptide or polypeptide comprising at least two domains that together specifically bind nucleic acid and one or more domains that specifically bind a paramagnetic metal, wherein one of the domains that bind the paramagnetic metal is between the domains that specifically bind nucleic acid.

24. A method for obtaining a magnetic resonance image of a mammal:
(a) administering to the mammal an effective amount of a magnetic resonance imaging contrast agent in a physiologically acceptable medium, the magnetic resonance imaging contrast agent comprising a synthetic peptide or polypeptide comprising at least two domains that together specifically bind nucleic acid and one or more domains that specifically bind a paramagnetic metal, wherein one of the domains that specifically bind the paramagnetic metal is between the domains that specifically bind nucleic acid; and
(b) recording the magnetic resonance image of the mammal.

25. A method for making magnetic resonance measurements of a sample of mammalian tissue by modifying the characteristic relaxation times of water protons in the sample, comprising:
(a) introducing to the sample a magnetic resonance imaging contrast agent comprising a synthetic peptide or polypeptide comprising at least two domains that together specifically bind nucleic acid and one or more domains that specifically bind a paramagnetic metal, wherein one of the domains that specifically bind the paramagnetic metal is between the domains that specifically bind nucleic acid;
(b) placing the sample in a magnetic field;
(c) providing a radio frequency pulse to the sample; and
(d) measuring the relaxation times.

26. A method for enhancing contrast in magnetic resonance images of a sample of mammalian tissue, comprising: introducing to the sample a magnetic resonance imaging contrast agent comprising a synthetic peptide or polypeptide comprising at least two domains that together specifically bind nucleic acid and one or more domains that specifically bind a paramagnetic metal, wherein one of the domains that specifically bind the paramagnetic metal is between the domains that specifically bind nucleic acid, and performing magnetic resonance imaging of the sample.

27. The method of any one of claims 24 to 26 wherein the mammalian tissue comprises a tumor.

28. The method of claim 27 wherein the mammalian tissue that comprises the tumor is located in the breast, lung, thyroid, lymph node, genitourinary system, musculoskeletal system, gastrointestinal tract, central or peripheral nervous system, head, neck or heart.

29. The method of claim 27 wherein the mammalian tissue that comprises the tumor is located in the kidney, urethra, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, or salivary gland.

30. The method of any one of claims 24 to 26 wherein the amount of magnetic resonance imaging contrast agent administered is about 0.025 mmol/kg of body weight, or less, inclusive.

31. The method of any one of claims 24 to 26 wherein the domains that specifically bind nucleic acid are from a transcription factor or a homeodomain.

32. The method of claim 31 wherein the domains that specifically bind nucleic acid comprise a helix-turn-helix motif, a relaxed helix-turn-helix motif, a winged helix-turn-helix motif, a helix-loop-strand motif, or a hormone receptor motif.

33. The method of anyone of claims 24 to 26 wherein the peptide or polypeptide comprises the amino acid sequence TERRRQQLDKDGDGTIDEREIKIWFQNKRAKIK (SEQ ID NO: 1), TERRRFDKDGNGYISAAELRHVKIWFQNKRAKIK (SEQ ID NO:3), TERRRFRVFDKDGNGYISAAEKIWFQNKRAKIK (SEQ ID NO:4), TRRRRFLSFDKDGDGTITTKEEVWFQNRRMKWK (SEQ ID NO:5) TERRRQQLDKDGDGTIDEREIKIWFQNKRAKIK (SEQ ID NO:7), TERRRQQLDKDGDGTIDEREQIKIWF (SEQ ID NO:10), DEKRPRTAFSGEQLARLKREFNENRYLTERRRLRVFDKDGNGFISAAEKI WFQNKRAKIKKST (SEQ ID NO:6), TERRRQQLSSEVGMTCSGCSGQIKIWF (SEQ ID NO:8), TERRRHELMHAIGFYHEAQIKIWF (SEQ ID NO:9) TERRRDIDGDGTITAKEKIWF (SEQ ID NO:13), TERRRQQLFDIDGDGTITAKEEAQIKIWF (SEQ ID NO:14), TERRRQQLSHGGGWGEAQIKIWFQNKRA (SEQ ID NO:15), or a portion thereof which specifically binds nucleic acid and specifically binds the paramagnetic metal.

34. The method of any of claims 24 to 26 wherein the peptide or polypeptide binds Gd(III), Fe(III), Mn(II), Mn(III), Dy(III), Yt(III), or Cr(III).

35. The method of any one of claims 24 to 26 wherein the peptide or polypeptide binds a lanthanide.

36. The method of any one of claims 24 to 26 wherein the magnetic resonance imaging contrast agent further comprises a protein transport domain.

37. The method of any one of claims 24 to 26 wherein the magnetic resonance imaging contrast agent further comprises a metal.

38. The method of any one of claims 24 to 26 wherein the magnetic resonance imaging contrast agent is bound to the metal.

39. The method of any one of claims 24 to 26 wherein the magnetic resonance imaging contrast agent is co-administered with the metal.

40. The method of one any of claims 37 to 39 wherein the metal is a lanthanide.

41. A kit comprising:
(a) a first container comprising a magnetic resonance imaging contrast agent comprising a synthetic peptide or polypeptide comprising at least two domains that together specifically bind nucleic acid and one or more domains that specifically bind a paramagnetic metal, wherein one of the domains that specifically binds the paramagnetic metal is between the domains that bind nucleic acid;
(b) a second container comprising a paramagnetic metal;
(c) a third container comprising a pharmaceutically acceptable carrier.

42. A kit comprising:
(a) a first container comprising a magnetic resonance imaging contrast agent comprising a synthetic peptide or polypeptide comprising at least two domains that together specifically bind nucleic acid and one or more domains that are bound to a paramagnetic metal, wherein one of the domains that is bound to the paramagnetic metal is between the domains that bind nucleic acid; and
(b) a second container comprising a pharmaceutically acceptable carrier.

43. The kit of any one of claims 41 to 42 further comprising instructions or printed indicia.

44. The kit of any one of claims 41 to 42 further comprising packaging material.
